(19) United States
(12) Patent Application Publication
(10) Pub. No.: US 2026/0231848 A1
(43) Pub. Date: Aug. 13, 2026

(54) A METHOD OF TREATING SEEDS AND SEEDS PRODUCED THEREBY

(71) Applicants: RESEARCH FOUNDATION OF THE CITY UNIVERSITY OF NEW YORK, New York, NY (US); CYPRUS UNIVERSITY OF TECHNOLOGY, Lemesos (CY)

(72) Inventors: Vasileios FOTOPOULOS, Limassol (CY); Chrsystalla ANTONIOU, Nicosia (CY); Khosrow KASHFI, Dix Hills, NY (US)

(73) Assignees: RESEARCH FOUNDATION OF THE CITY UNIVERSITY OF NEW YORK, New York, NY (US); CYPRUS UNIVERSITY OF TECHNOLOGY, Lemesos (CY)

(21) Appl. No.: 19/156,621

(22) PCT Filed: Feb. 15, 2024
(86) PCT No.: PCT/US2024/015951

§ 371 Date: Aug. 14, 2025

Related U.S. Application Data
(60) Provisional application No. 63/446,079, filed on Feb. 16, 2023.

(51) Int. Cl. A01C 1/06; C07C 311/46; C07C 321/14; C07D 207/50; C07D 271/08; C07D 339/04; C07D 409/12; C07D 413/12

(52) U.S. Cl. CPC A01C 1/06; C07C 311/46; C07C 321/14; C07D 207/50; C07D 271/08; C07D 339/04; C07D 409/12; C07D 413/12; C07C 2601/16

(57) ABSTRACT

Methods of treating seeds to promote plant growth from the seeds and to induce tolerance to stress conditions including exposing seeds to an aqueous composition comprising a compound containing an NO-releasing moiety and an H₂S-releasing moiety covalently bonded together, a compound containing an NO-releasing moiety and an H₂S-releasing moiety covalently bonded to an aspirin derived core, or combinations thereof; and a polar solvent, when the seeds are pre-germination. The seeds and a kit containing the compound(s) is also claimed.

### TECHNOLOGICAL FIELD

The present disclosure relates to agriculture and specifically to methods for treating seeds.

### BACKGROUND OF THE INVENTION

There is a growing need to improve plant yield and productivity in existing agricultural lands.

NOSH-ASA, a hybrid synthetic compound containing an H₂S releasing moiety and a nitric oxide (NO) releasing moiety covalently linked with a salicylic acid moiety was first described for treating inflammatory diseases and cancer (Chattopadhyay et al. 2012).

In addition, International Patent Application Publication No. WO 2013/025790 described the use of various such compounds containing the H₂S releasing moiety and the nitric oxide (NO) releasing moiety covalently linked with a core, including a salicylic acid moiety, for the treating of inflammatory diseases and cancer.

Later, International Patent Publication No. 2015/123273 described the use of compounds containing an H₂S-releasing moiety and NO-releasing moiety covalently bonded together and/or a compound containing an H₂S-releasing moiety and NO-releasing moiety covalently linked to a core, for example an aspirin derivative, for promoting plant growth and priming plants against abiotic stress factors by treating the plants.

International Patent Publication No. WO 2013/025790 and International Patent Publication No. WO 2015/123273 are herein incorporated by reference in their entirety.

### SUMMARY OF THE INVENTION

The present disclosure provides, in accordance with a first of its aspects, a method of treating seeds, the method comprises exposing said seeds to an aqueous composition comprising a compound containing an NO-releasing moiety and an H₂S-releasing moiety covalently bonded together, a compound containing an NO-releasing moiety and an H₂S-releasing moiety covalently bonded to an aspirin derived core, or combinations thereof; wherein said exposing of the seeds to said aqueous composition is when the seeds are at their pre-germination stage.

In a further aspect, the present disclosure provides seeds comprising an amount of the compound(s), wherein the seeds are at their pre-germinated stage.

In yet a further aspect, the present disclosure provides a kit for treating seeds, the kit includes the compound(s) and/or an aqueous composition comprising the compound(s) and instruction for use of same for exposing seeds to the compound(s) when the seeds are at their pre-germination stage.

In one embodiment, the invention relates to a method of treating seeds to promote plant growth from the seeds comprising exposing seeds to an aqueous composition comprising a compound containing an NO-releasing moiety and an H₂S-releasing moiety covalently bonded together, a compound containing an NO-releasing moiety and an H₂S-releasing moiety covalently bonded to an aspirin derived core, or combinations thereof; and a polar solvent, when the seeds are pre-germination.

In another embodiment, the invention relates to a method of treating seeds to induce tolerance to stress conditions comprising exposing seeds to an aqueous composition comprising a compound containing an NO-releasing moiety and an H₂S-releasing moiety covalently bonded together, a compound containing an NO-releasing moiety and an H₂S-releasing moiety covalently bonded to an aspirin derived core, or combinations thereof; and a polar solvent, when the seeds are pre-germination.

Preferably, the compound containing an NO-releasing moiety and an H₂S-releasing moiety covalently bonded together is of formula II:

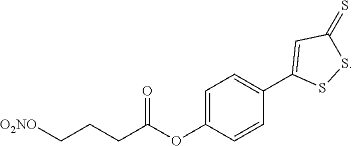

In another preferred embodiment, the compound containing an NO-releasing moiety and an H₂S-releasing moiety covalently bonded to an aspirin derived core is of formula I:

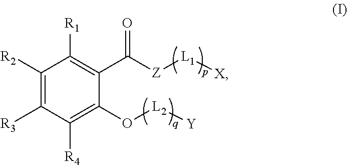

wherein:
each of p and q, independently, is 0 or 1;
each of L₁ and L₂, independently, is a linker, the linker being —C(O)—, —(CH₂)ₘ—, —(CH₂)ₘ—O—, —(CH₂)ₘ—C(O)—, —(CH₂)ₘ—C(O)O—, —(CH₂)ₘ—OC(O)O—, —C(O)—(CH₂)ₘ—O—, —C(O)—(CH₂)ₘ—C(O)—, —OC(O)—(CH₂)ₘ—O—, —OC(O)—(CH₂)ₘ—C(O)—, or —OC(O)—(CH₂)ₘ—C(O)O—, in which m is 1, 2, 3, 4, 5, 6, or 7;
X is a H₂S-releasing moiety or a NO-releasing moiety;
Y is a NO-releasing moiety or a H₂S-releasing moiety, provided that X and Y are not simultaneously H₂S-releasing moieties or NO-releasing moieties;
Z is O or NH; and
each of R₁, R₂, R₃, and R₄, independently, is H, halo, C₁-C₁₀ alkyl, or N(R)₂, in which R is H or C₁-C₁₀ alkyl.
wherein
the H₂S-releasing moiety is

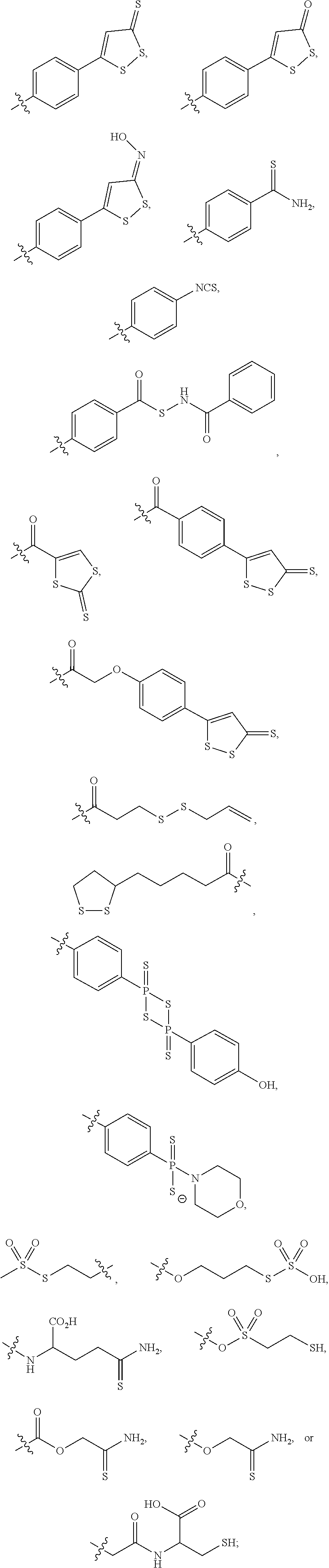

and
the NO-releasing moiety is —NO, —C(O)—(CH₂)ₙ—ONO₂, —O—(CH₂)ₙ—ONO₂, —(CH₂)—ONO₂, —C(O)—CH₂—C(CH₃)₂—SNO, —NH—CH₂—C(CH₃)₂—SNO, —CH₂—C(CH₃)₂—SNO,

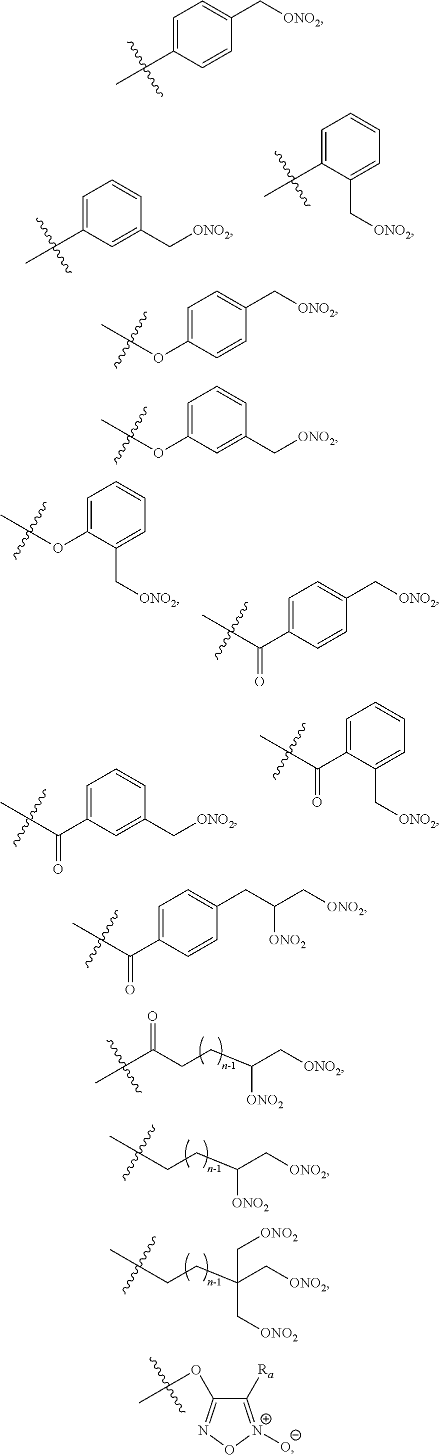

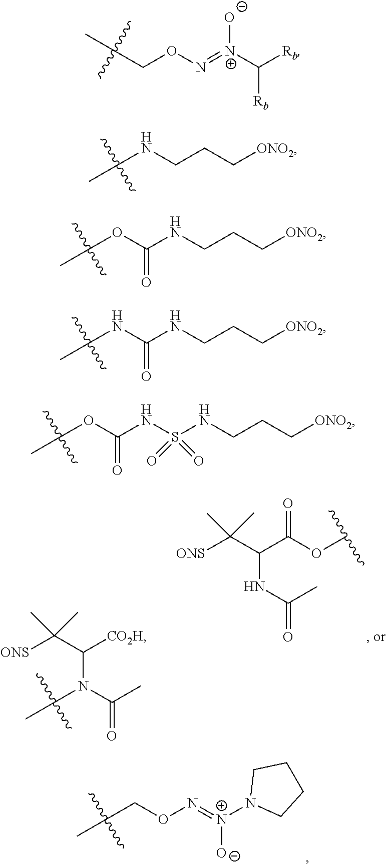

in which n is 1, 2, 3, 4, 5, 6, or 7;
Rₐ is H, C₁-C₁₀ alkyl, aryl, S(O)₂-aryl, CN, or CON(R_{b})₂; and each R_{b}, independently, is H or C₁-C₁₀ alkyl.

Preferably, exposing the seeds comprises soaking the seeds in the aqueous composition. The compound or compounds are preferably present in a concentration of up to about 100 μM in the aqueous composition. More preferably, the compound or compounds are present in a concentration of from about 1 μM to about 100 μM. Most preferably, the compound or compounds are present in a concentration of from about 10 μM to about 50 μM.

The polar solvent is preferably selected from the group consisting of dichloromethane (DCM), tetrahydrofuran (THF), ethyl acetate, acetonitrile, dimethylformamide (DMF), dimethyl sulfoxide (DMSO), acetone, methanol, ethanol, isoproplyl alcohol, acetic acid, and hexamethylphosphoric triamde (HMPT).

The polar solvent is preferably a polar aprotic solvent. The most preferred polar solvent is DMSO.

Preferably the polar solvent is in a concentration of about 0.01% v/v to about 15% v/v. Preferably, the aqueous composition comprises about 0.1% v/v±0.05% v/v DMSO.

There are typically one or more treatments of the seeds prior to germination. Preferably, the treatment is devoid of subsequent exposure of the seeds after germination or a plant grown from said seeds to a NOSH compound.

The aqueous composition is made by
a. dissolving the compound or compounds in an analytical grade polar solvent; and
b. diluting the dissolved compound or compounds with water to obtain an aqueous composition comprising the dissolved compound or compounds.

Pre-germinated seeds comprising a compound containing an NO-releasing moiety and an H₂S-releasing moiety covalently bonded together, a compound containing an NO-releasing moiety and an H₂S-releasing moiety covalently bonded to an aspirin derived core, or combinations thereof. The pre-germinated seeds, wherein the compound containing an NO-releasing moiety and an H₂S-releasing moiety covalently bonded together is of formula II:

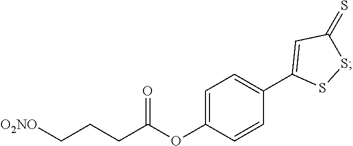

and
the compound containing an NO-releasing moiety and an H₂S-releasing moiety covalently bonded to an aspirin derived core is of formula I:

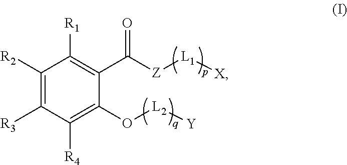

wherein:
each of p and q, independently, is 0 or 1;
each of L₁ and L₂, independently, is a linker, the linker being —C(O)—, —(CH₂)ₘ—, —(CH₂)ₘ—O—, —(CH₂)ₘ—C(O)—, —(CH₂)ₘ—C(O)O—, —(CH₂)ₘ—OC(O)O—, —C(O)ₘCH₂)ₘ—O—, —C(O)—(CH₂)ₘ—C(O)—, —OC(O)—(CH₂)ₘ—O—, —OC(O)—(CH₂)ₘ—C(O)—, or —OC(O)—(CH₂)ₘ—C(O)O—, in which m is 1, 2, 3, 4, 5, 6, or 7;
X is a H₂S-releasing moiety or a NO-releasing moiety;
Y is a NO-releasing moiety or a H₂S-releasing moiety, provided that X and Y are not simultaneously H₂S-releasing moieties or NO-releasing moieties;
Z is O or NH; and
each of R₁, R₂, R₃, and R₄, independently, is H, halo, C₁-C₁₀ alkyl, or N(R)₂, in which R is H or C₁-C₁₀ alkyl,
wherein
the H₂S-releasing moiety is

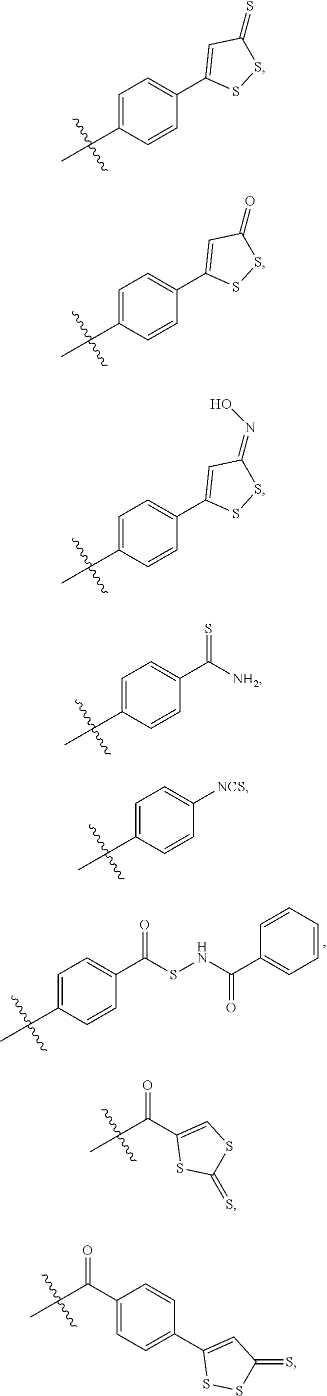

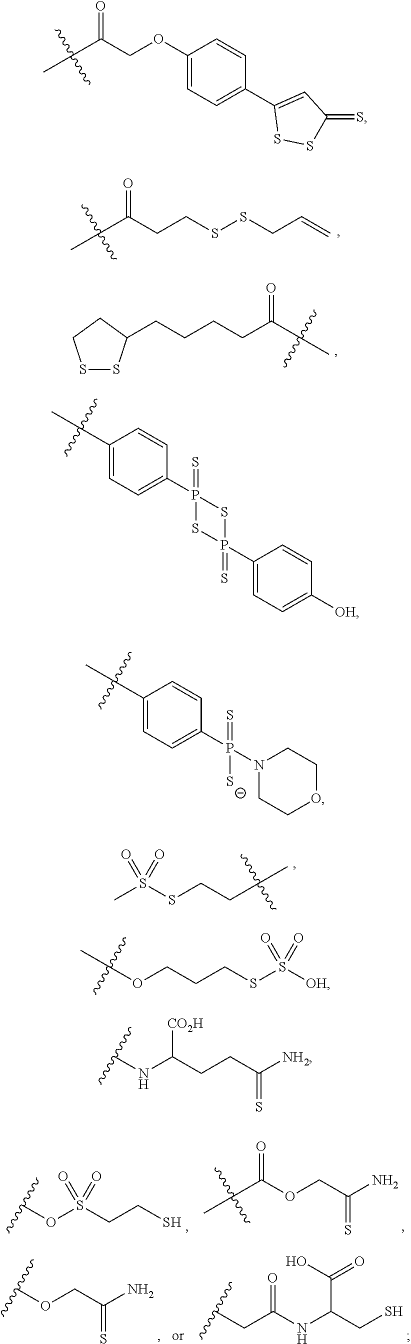

and
the NO-releasing moiety is —NO, —C(O)—(CH₂)ₙ—ONO, —O—(CH₂)ₙ—ONO₂, —(CH₂)ₙ—ONO₂, —C(O)—CH₂—C(CH₃)₂—SNO, —NH—CH₂—C(CH₃)₂—SNO, —CH₂—C(CH₃)₂—SNO,

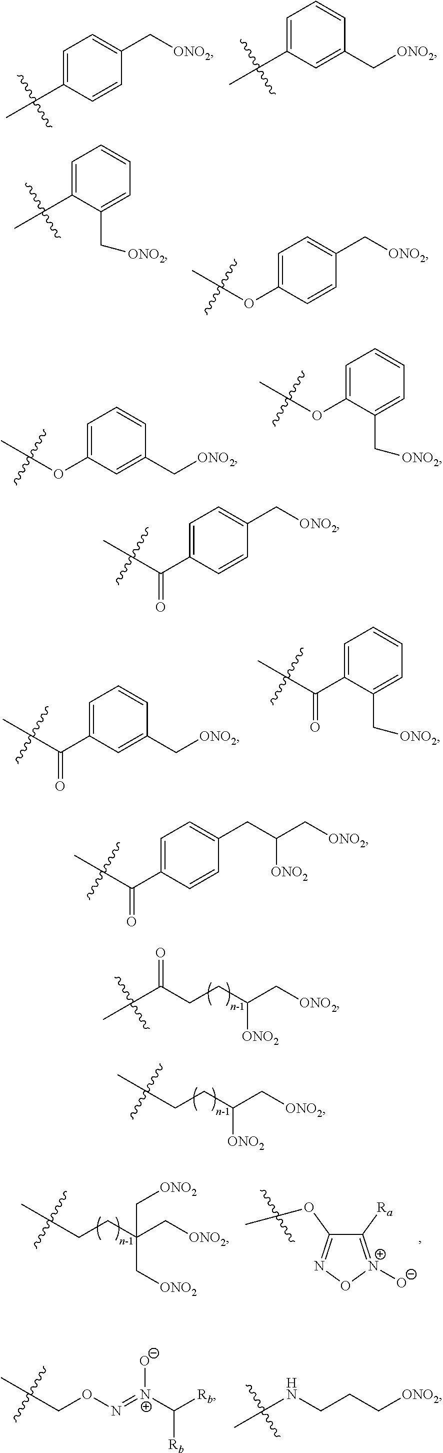

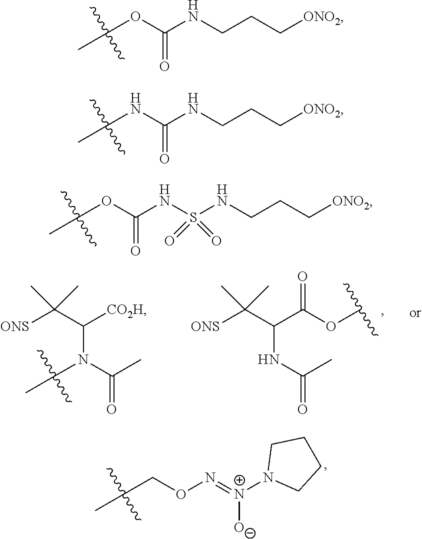

in which n is 1, 2, 3, 4, 5, 6, or 7;
Rₐ is H, C₁-C₁₀ alkyl, aryl, S(O)₂-aryl, CN, or CON(R_{b})₂; and each R_{b}, independently, is H or C₁-C₁₀ alkyl.

In another embodiment, the invention relates to a kit comprising an NO-releasing moiety and an H₂S-releasing moiety covalently bonded together, a compound containing an NO-releasing moiety and an H₂S-releasing moiety covalently bonded to an aspirin derived core, or combinations thereof; and a solvent for dissolving the same, wherein compound or compounds are in dry form or dissolved in the solvent; and instructions for use of the compounds or compounds in dissolved form for treating seeds at their pre-germination stage.

The kit preferably includes instructions for preparing an aqueous solution including the compounds or compounds in concentrations effective from treating the seeds. The kit also comprises a stock solution of the compound or compounds and instructions for diluting the stock solution with an aqueous medium to obtain an aqueous solution suitable for treating the seeds.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1B are bar graphs showing the effect of seed treatment with different concentrations of NOSH or NOSH-ASA on the plant growth, where FIG. 1A presents the ratio between fresh weight of rosettes 22 days after stratification (DAS) from which the seeds treated with NOSH compounds compared with the rosettes from which seeds were treated with the carrier only (0.1% DMSO); while FIG. 1B presents the ratio of 22 DAS projected rosette area (PRA) of plants arising from NOSH compound-treated seeds compared with the carrier-treated seeds.

FIGS. 2A-2B present images of representative phenotype of Arabidopsis plants which the seeds were carrier-treated (treated only with the carrier being 0.1% DMSO) (FIG. 2A) or following treatment, at seed level, with 50 μM NOSH (FIG. 2B).

FIG. 3 is a bar graph showing the ratio between fresh weight of rosettes or PRA of plants treated at seed level with 50 μM NOSH and that of carrier-treated seeds.

FIG. 4 is a bar graph showing the ratio between (i) leaf area; (ii) pavement cell area; (iii) pavement cell number; and (iv) stomata index as measured on leaf 3 of plants treated at seed level with 50 μM NOSH, and the same parameter obtained for carrier-treated-seeds.

FIG. 5 shows fruit yield/plant of tomato plants (a), fruit fresh weight (b) and fruit dry weight (c) under control and high salinity conditions.

FIGS. 6(a)-(d) shows seeds shown in commercial greenhouse (a); survival percentages of the various plants (b); weights and total plant leaf area (c); and average leaf area and number of leaves (d).

### DETAILED DESCRIPTION OF THE INVENTION

The invention relates to a method of treating seeds to promote plant growth from the seeds and to induce tolerance to stress conditions including exposing the seeds to an aqueous composition comprising a compound containing an NO-releasing moiety and an H₂S-releasing moiety covalently bonded together, a compound containing an NO-releasing moiety and an H₂S-releasing moiety covalently bonded to an aspirin derived core, or combinations thereof; and apolar solvent, when the seeds are pre-germination.

The seeds are preferably in the pre-germination stage of development. The pre-germination stage is also known as the pre-sprouting stage of a seed.

The compound containing an NO-releasing moiety and an H₂S-releasing moiety covalently bonded together does not contain a linker. The NO-releasing moiety and an H₂S-releasing moiety are directly bonded together. An example of an NO-releasing moiety and an H₂S-releasing moiety covalently bonded together is formula II below, the compound known as NOSH.

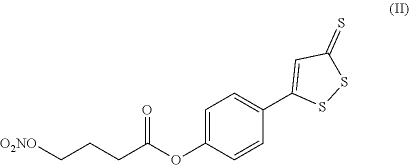

Preferably, the compound containing an NO-releasing moiety and an H₂S-releasing moiety covalently bonded to an aspirin derived core is of formula I:

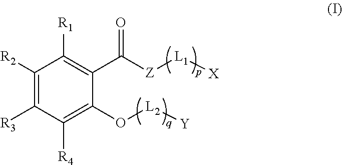

wherein each of p and q, independently, is 0 or 1; each of L₁ and L₂, independently, is a linker, the linker being —C(O)—, —(CH₂)ₘ, —(CH₂)ₘ—O—, —(CH₂)ₘ—C(O)—, —(CH₂)ₘ—C(O)O—, —(CH₂)ₘ—OC(O)O—, —C(O)—(CH₂)ₘ—O—, —C(O)—(CH₂)ₘ—C(O)—, —OC(O)—(CH₂)ₘ—O—, —OC(O)—(CH₂)ₘ—C(O)—,or —OC(O)—(CH₂)ₘ—C(O)O—, in which m is 1, 2, 3, 4, 5, 6, or 7; X is a H₂S-releasing moiety or a NO-releasing moiety; Y is a NO-releasing moiety or a H₂S-releasing moiety, provided that X and Y are not simultaneously H₂S-releasing moieties or NO-releasing moieties; Z is O or NH; and each of R₁, R₂, R₃, and R₄, independently, is H, halo, C₁-C₁₀ alkyl, or N(R)₂, in which R is H or C₁-C₁₀ alkyl.

In a subset of the compounds of formula (I), X can be

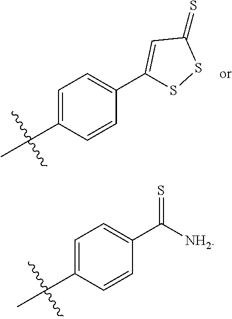 [SMILES: *c1ccc(-c2cc(=S)ss2)cc1 ; "or" ; *c1ccc(C(N)=S)cc1]

In some embodiments of such compounds, Y can be —C(O)—(CH₂)ₙ—ONO₂, and p and q can be 0. Examples of such compounds are

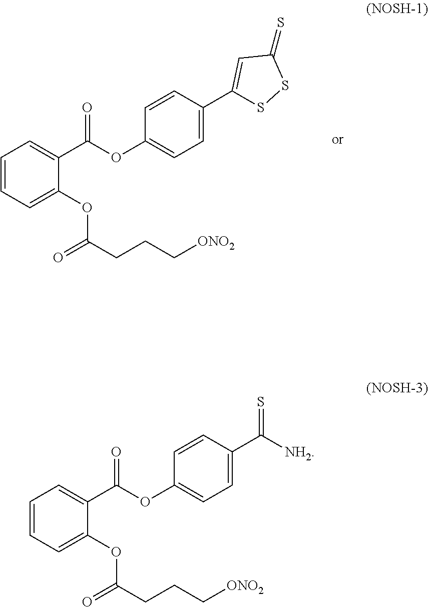

NOSH-1 is also referred to herein as NOSH-aspirin, NOSH-A, or NOSHA.

In some embodiments of such compounds, Y can be —(CH₂)ₙ—ONO₂, p can be 0, q can be 1, and L₂ can be —OC(O)—(CH₂)ₘ—C(O)—. An example of such a compound is

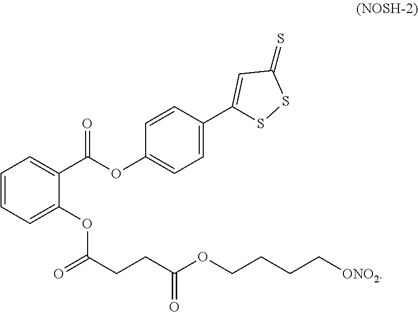

In another subset of the compounds of formula (I), X can be —C(O)—(CH₂)ₙ—ONO₂. In such a compound, Y can be

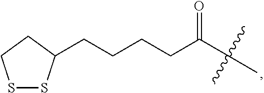 [SMILES: *C(=O)CCCCC1CCSS1]

and p and q can be 0. An example of such compounds is

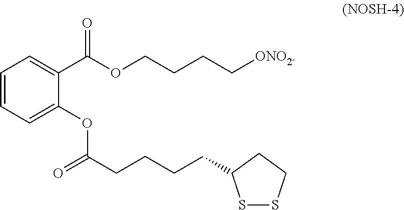

Other examples of the compounds of formula (I) include:

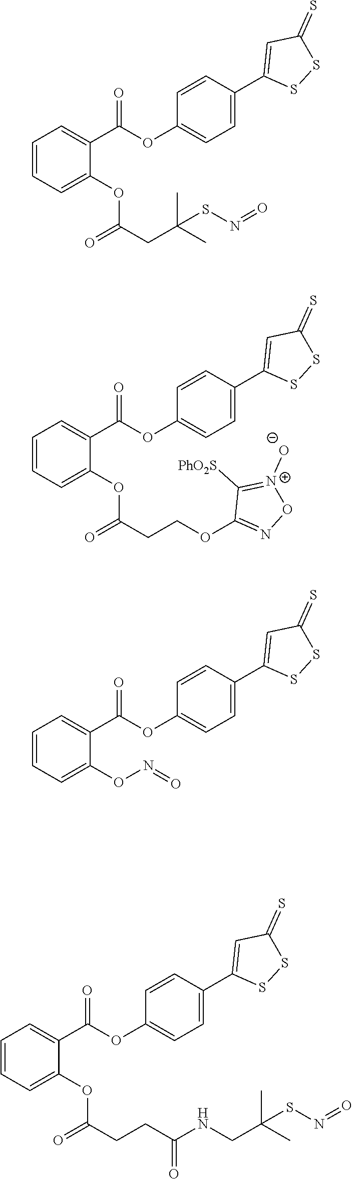

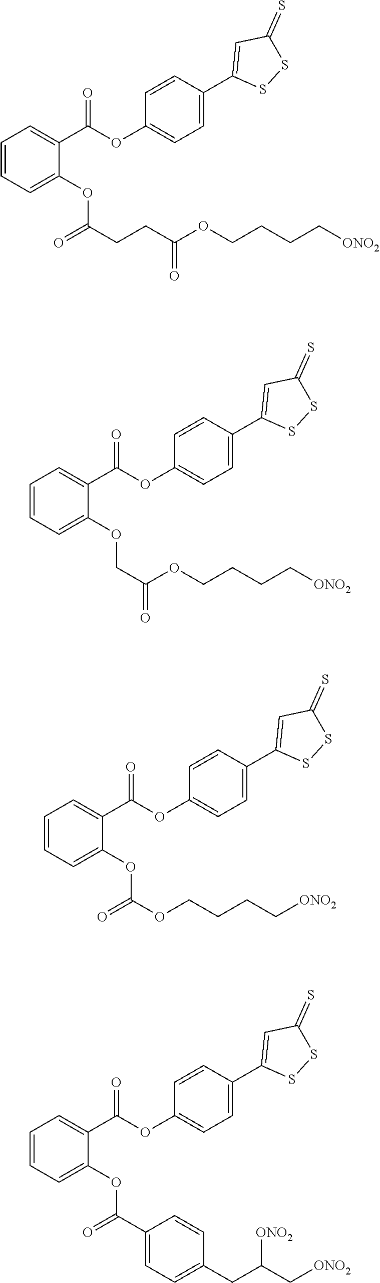

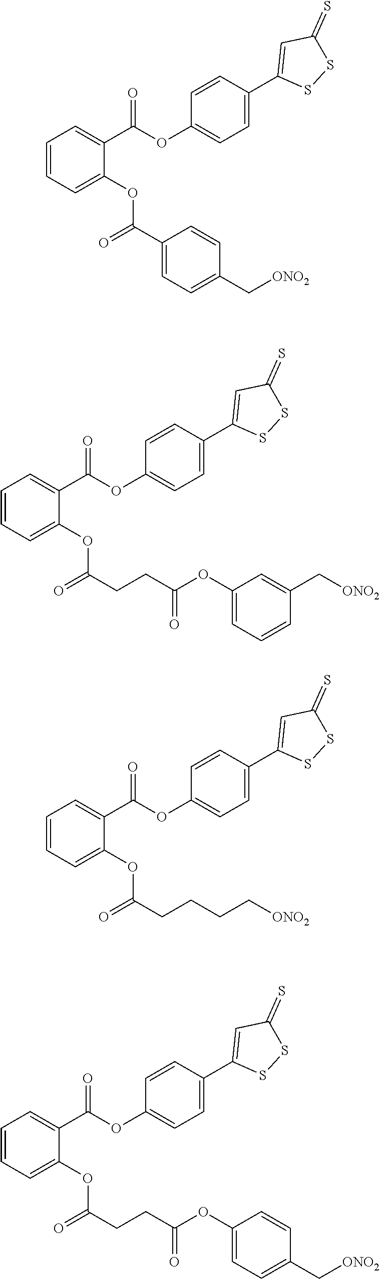

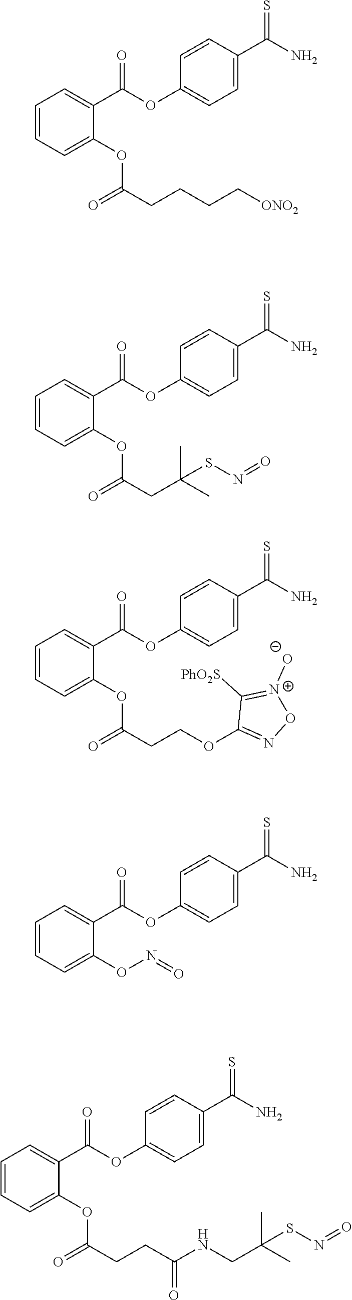

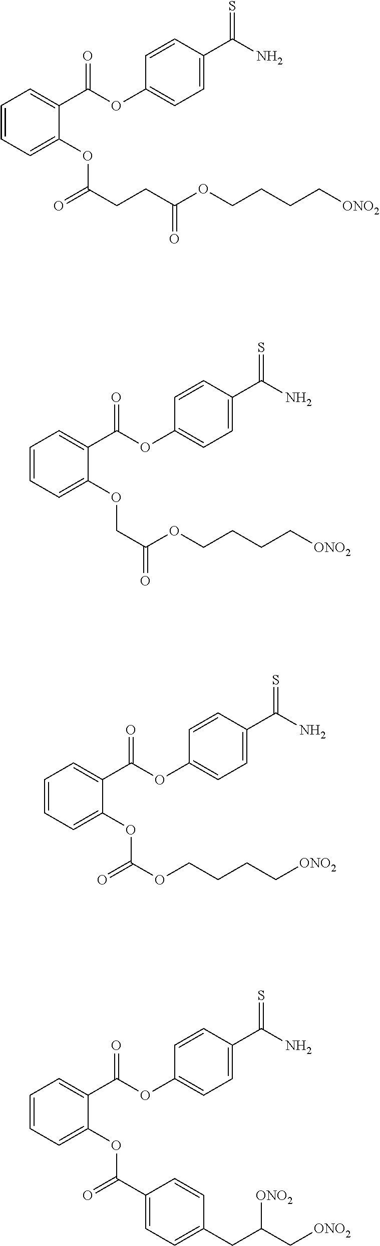

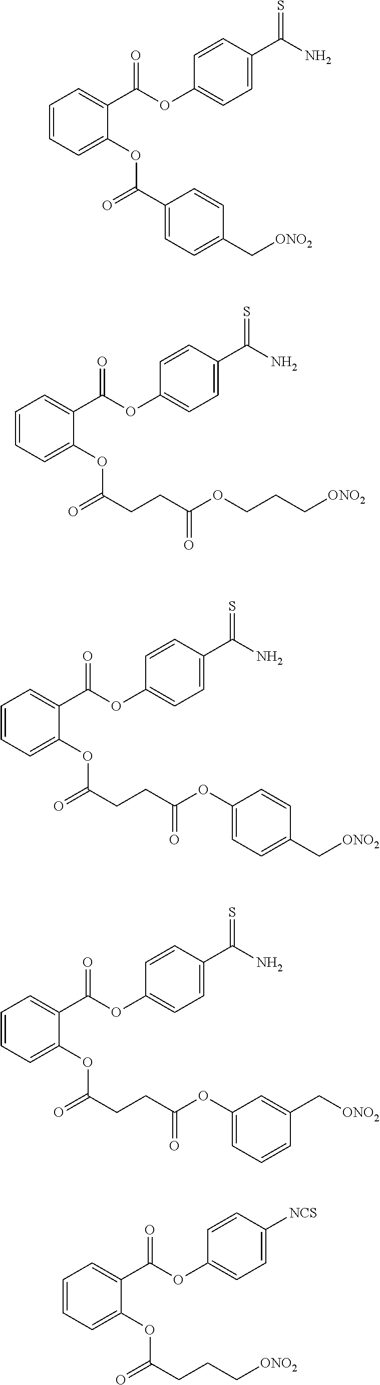

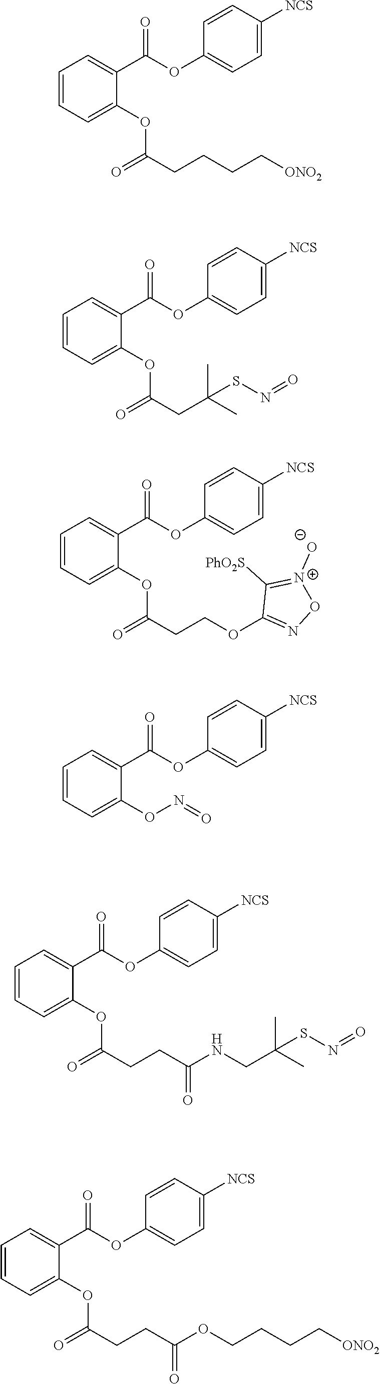

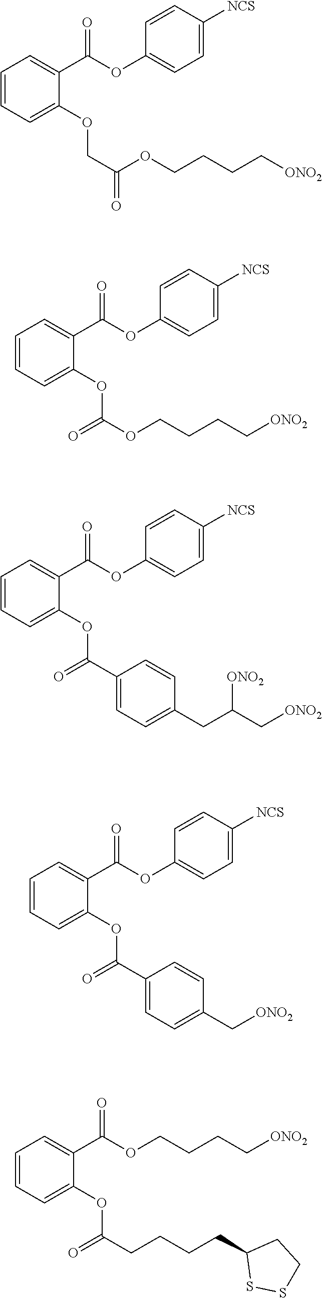

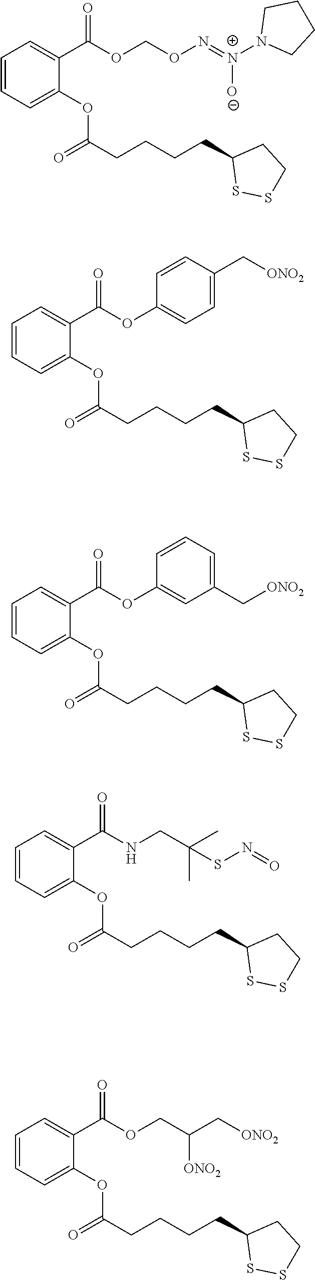

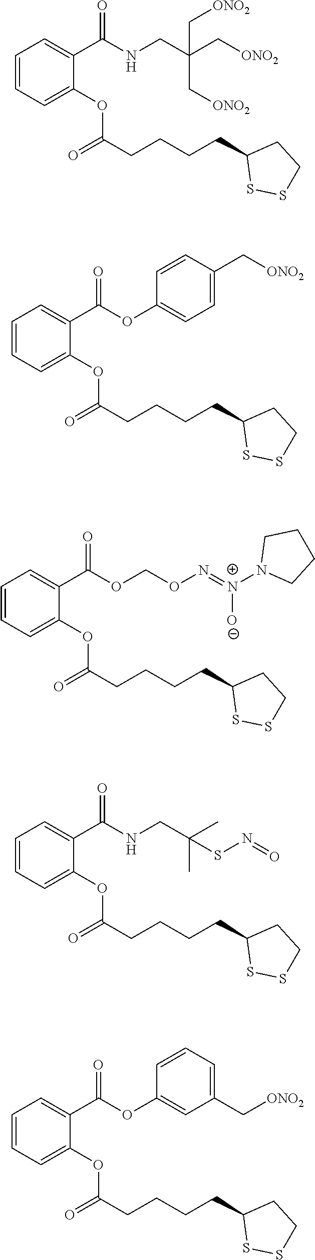

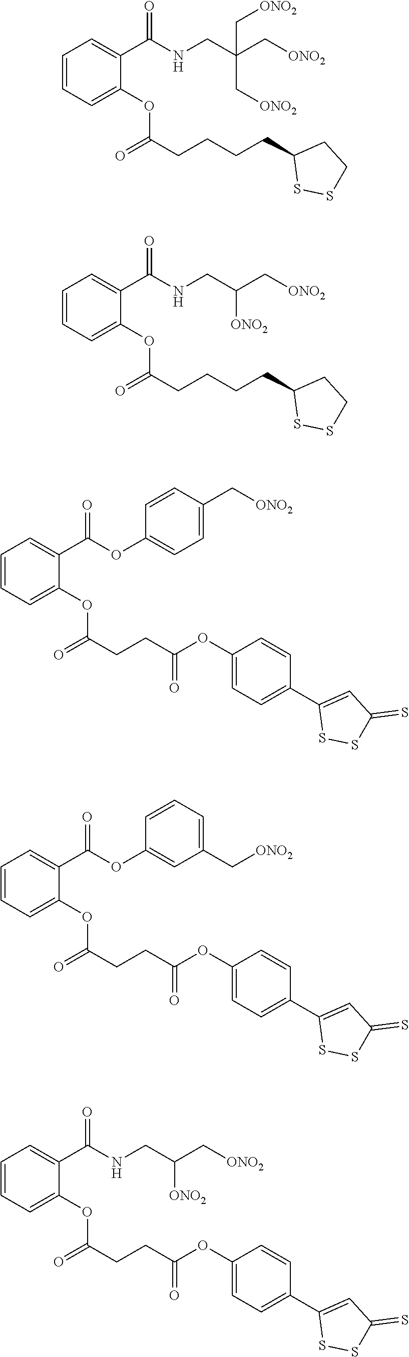

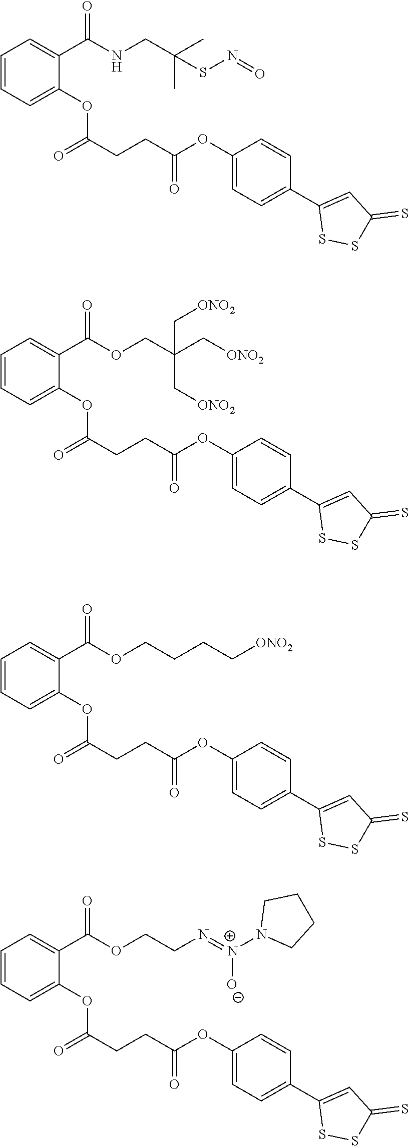

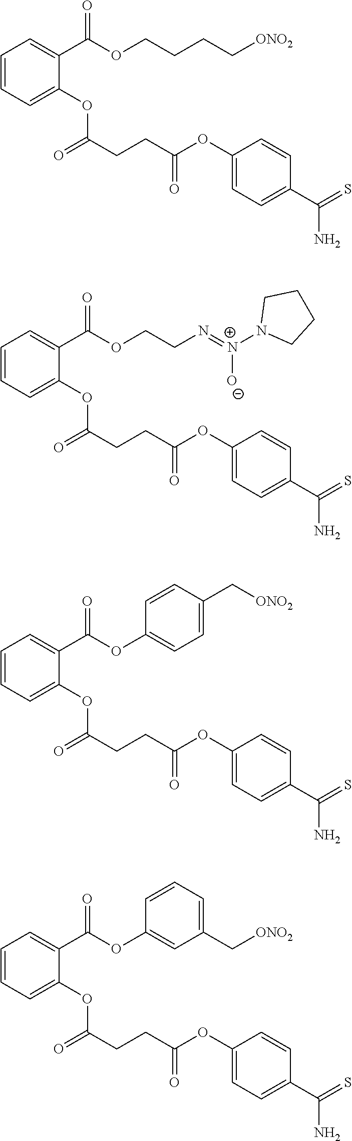

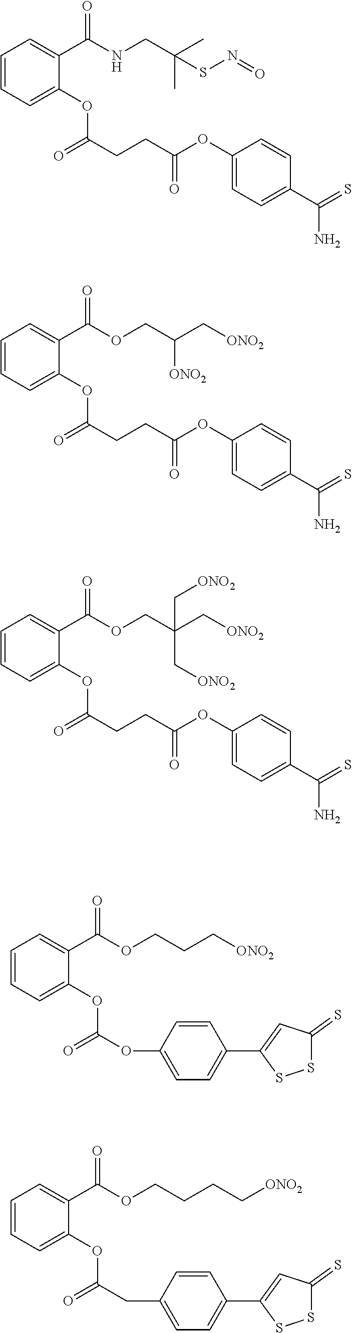

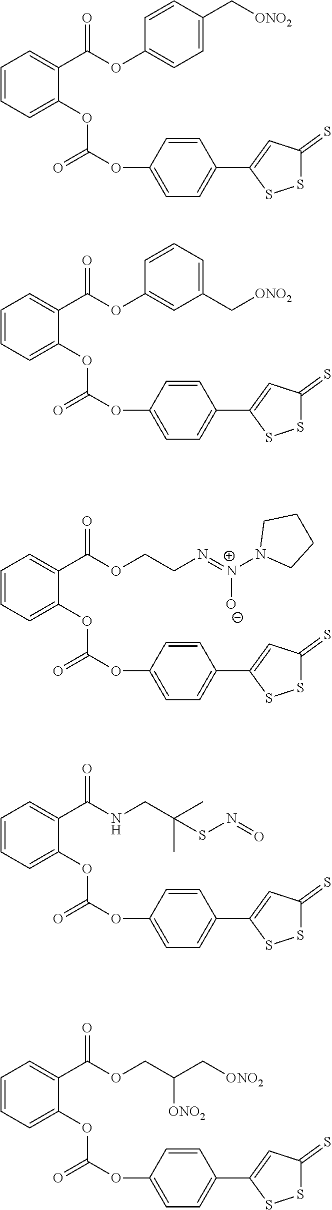

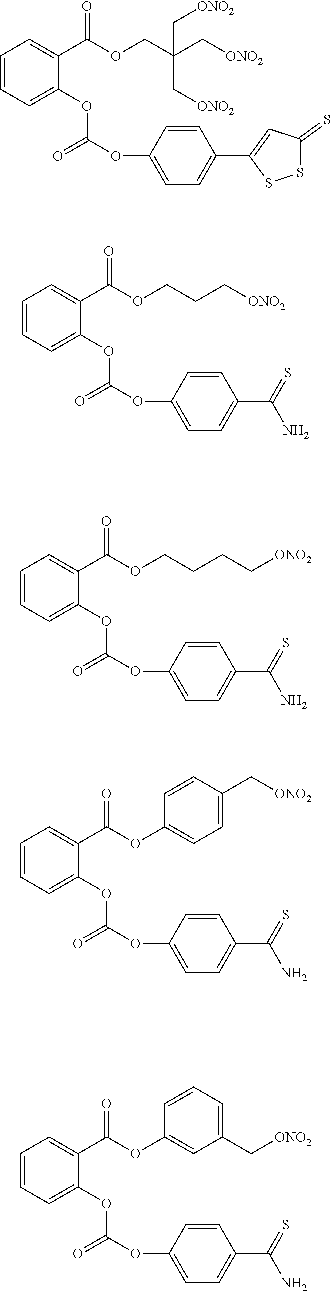

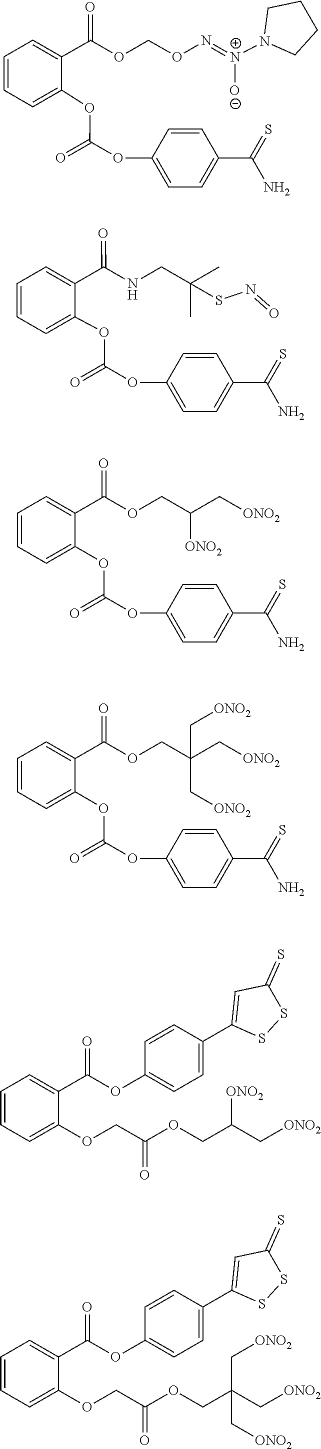

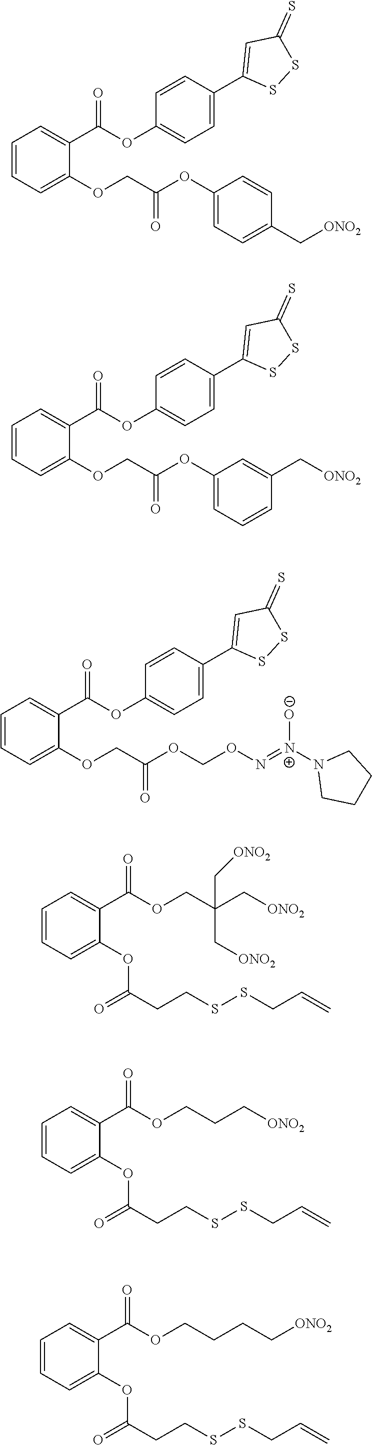

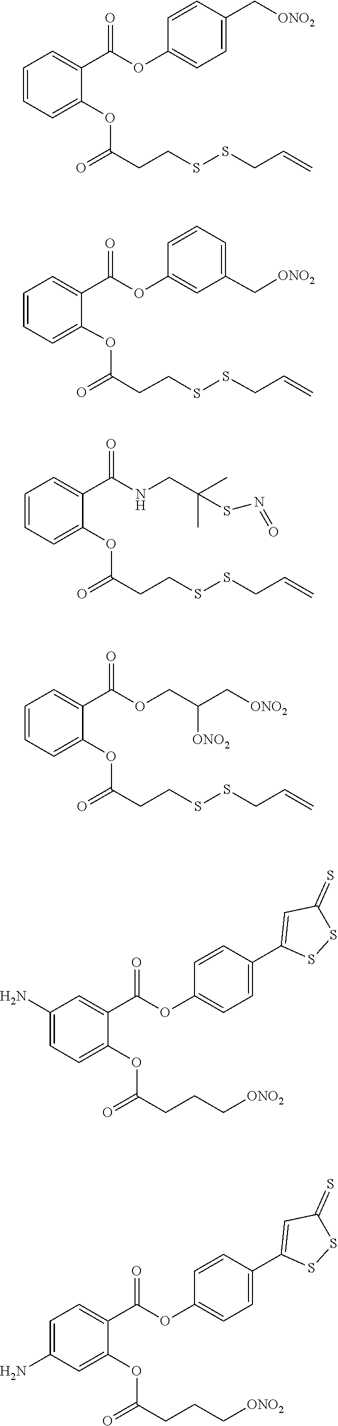

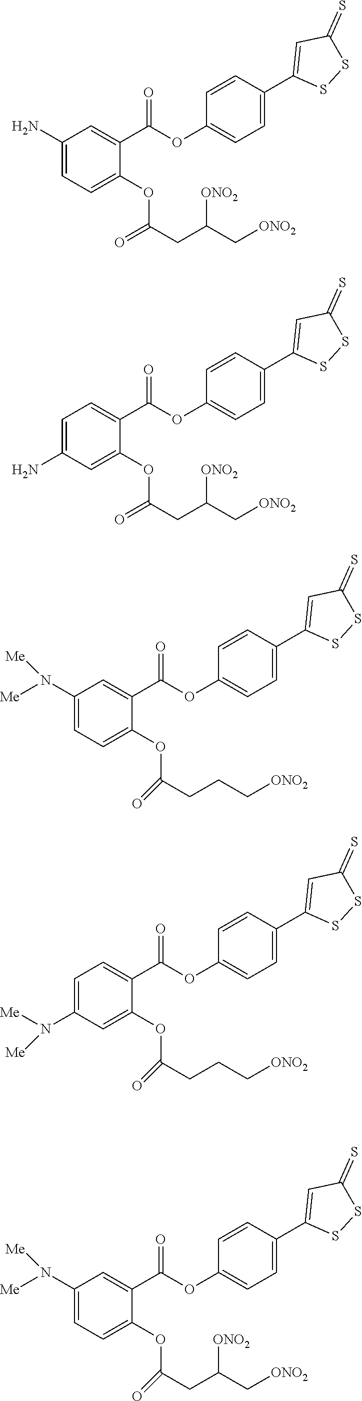

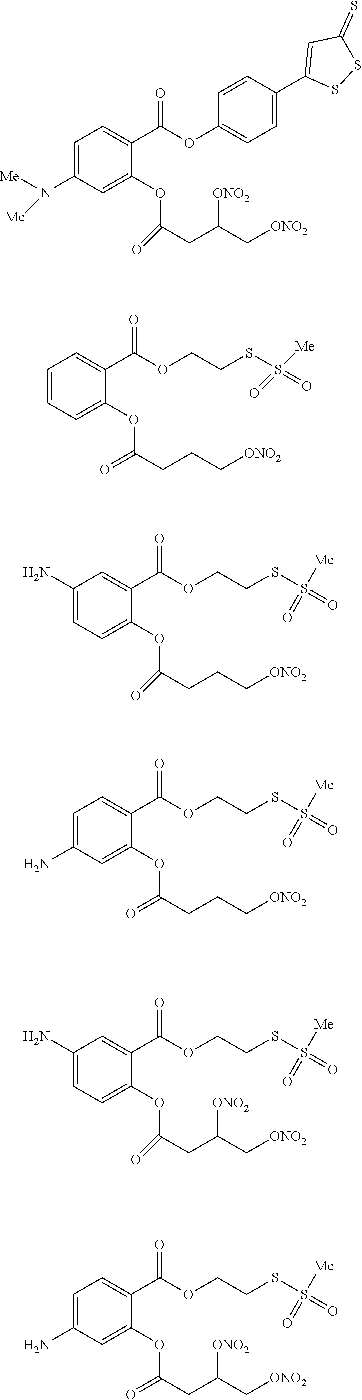

As used herein, “a NO-releasing moiety” refers to a moiety that can be cleaved from a parent compound to generate NO under physiological conditions after the parent compound is administered to a patient. Examples of suitable NO-releasing moieties include —NO, —C(O)—(CH₂)ₙ—ONO₂, —O—(CH₂)ₙ—ONO₂, —(CH₂)ₙ—ONO₂, —C(O)—CH₂—C(CH₃)₂—SNO, —NH—CH₂—C(CH₃)₂—SNO, —CH₂—C(CH₃)₂—SNO,

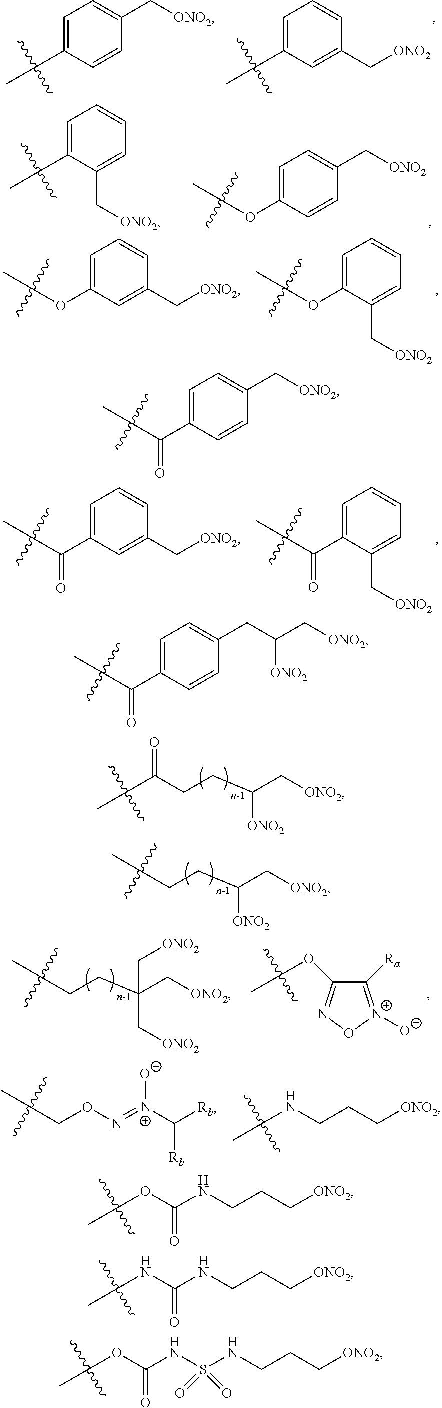

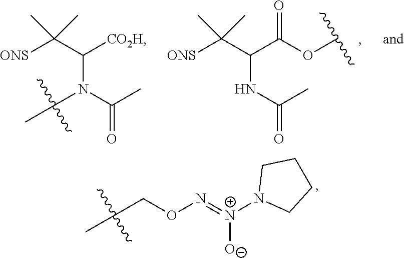

in which n is 1, 2, 3, 4, 5, 6, or 7; Rₐ is H, C₁-C₁₀ alkyl, aryl, S(O)₂-aryl, CN, or CON(R_{b})₂; and each R_{b}, independently, is H or C₁-C₁₀ alkyl.

The term “alkyl” refers to a saturated, linear or branched hydrocarbon moiety, such as —CH₃ or —CH(CH₃)₂. The term “aryl” refers to a hydrocarbon moiety having one or more aromatic rings. Examples of aryl moieties include phenyl (Ph), naphthyl, pyrenyl, anthryl, and phenanthryl. Alkyl and aryl mentioned herein include both substituted and unsubstituted moieties, unless specified otherwise. Possible substituents on aryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino. C₁-C₁₀ alkylamino, C₁-C₂₀ dialkylamino, arylamino, diarylamino, C₁-C₁₀ alkylsulfonamino, arylsulfonamino, C₁-C₁₀ alkylimino, arylimino. C₁-C₁₀ alkylsulfonimino, arylsulfonimino, hydroxyl, halo, thio, C₁-C₁₀ alkylthio, arylthio, C₁-C₁₀ alkylsulfonyl, arylsulfonyl, arylsulfonamide, heteroarylsulfonamide, acylamino, aminoacyl, aminothioacyl, amidino, guanidine, ureido, cyano, nitro, nitroso, azido, acyl, thioacyl, acyloxy, carboxyl, and carboxylic ester. On the other hand, possible substituents on alkyl include all of the above-recited substituents except C₁-C₁₀ alkyl.

As used herein, “a H₂S-releasing moiety” refers to a moiety that can be cleaved from a parent compound to generate H₂S under physiological conditions after the parent compound is administered to a patient. Examples of suitable H₂S-releasing moieties include:

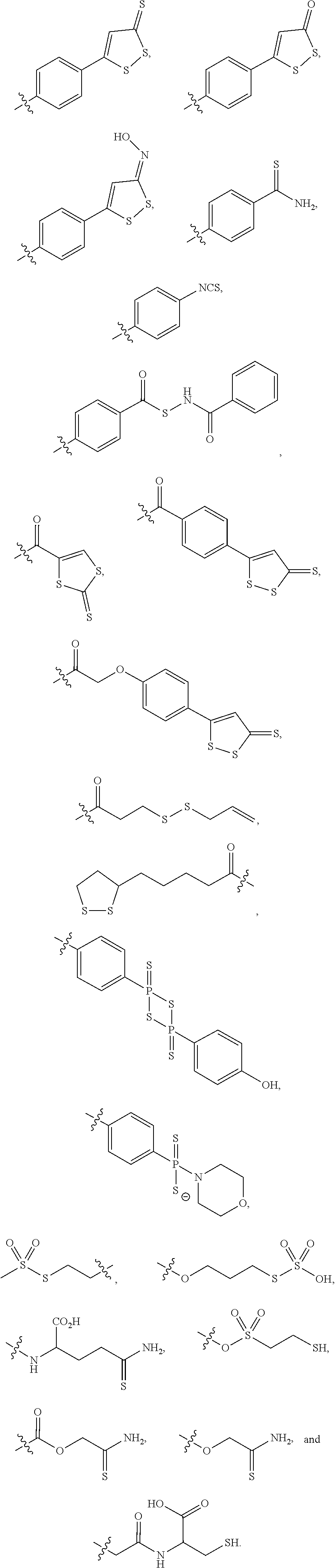

In another embodiment of the invention, the compound may be NOSH, an NO-releasing moiety and an H₂S-releasing moiety without an NSAID-derived core. The structure of NOSH is shown below:

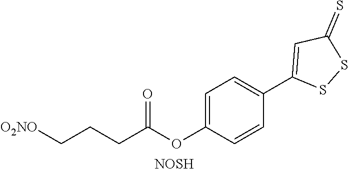

The compounds of the invention may be synthesized by known methods in the art. For example, see International Patent Publication No. WO 2013/025790, which discusses synthesis of the compounds.

The compound or compounds are present in a concentration of up to about 100 μM in the aqueous composition, i.e., 1 μM, 2 μM, 3 μM, 4 μM, 5 μM, 6 μM, 7 μM, 8 μM, 9 μM, 10 μM, 11 μM, 12 μM, 13 μM, etc. . . . The numerals between 1 and 100 may also be used to create a minima or maxima of ranges of number specifying the concentration of the compound or compounds in the aqueous solution in μM. For example, the compound or compounds are preferably present in a concentration of from about 1 μM to about 100 μM. Most preferably, the compound or compounds are present in a concentration of from about 10 μM to about 50 μM. The compound or compounds may be present in a concentration of any number between ranges.

The seeds may be exposed to the aqueous composition in any known manner such as spraying, soaking, etc. Preferably, exposing the seeds involves soaking the seeds in the aqueous composition for an adequate period of time. The soaking time may be, anywhere from a few seconds to a few hours so long as the compound(s) adhere to the seed and the seeds are not damaged. Soaking time depends upon the type of seeds being soaked, the compound(s), the concentration of the compound(s), the type of solvent, temperature, pH of the solution, etc.

The aqueous composition is made by dissolving the compound or compounds in an analytical grade polar solvent; and diluting the dissolved compound or compounds with water to obtain an aqueous composition comprising the dissolved compound or compounds.

The polar solvent is preferably selected from the group consisting of dichloromethane (DCM), tetrahydrofuran (THF), ethyl acetate, acetonitrile, dimethylformamide (DMF), dimethyl sulfoxide (DMSO), acetone, methanol, ethanol, isoproplyl alcohol, acetic acid, and hexamethylphosphoric triamde (HMPT). Preferably, the polar solvent is a polar aprotic solvent such as DMSO.

The polar solvent is preferably in a concentration of about 0.01% v/v to about 15% v/v (by weight of the aqueous composition). More preferably, the aqueous composition includes about 0.1% v/v±0.05% v/v of the polar solvent by weight of the total aqueous composition.

The seeds may undergo the treatment one or more times prior to germination. It is contemplated that the seeds will not be subsequently exposed after germination to the compounds described above and that the plant grown from said seeds will also not be exposed to the compounds described above.

Another embodiment of the invention relates to the seeds treated using the methods described above. The pre-germinated seeds treated by the above method are claimed.

Another embodiment of the invention relates to a kit including the compound(s) and a solvent for dissolving the compound(s), where the compound(s) are either in dry form or dissolved in the solvent; and instructions for use of the compound(s) in dissolved form for treating the seeds at their pre-germination stage. The kit may also contain instructions for preparing an aqueous solution including the compound(s) in concentrations effective from treating the seeds. The kit may also include a stock solution of the compound(s) and instructions for diluting the stock solution with an aqueous medium to obtain an aqueous solution suitable for treating seeds.

The present disclosure is based on the finding that treatment of seeds (of Arabidopsis thaliana) before germination with NOSH compounds as defined herein below, significantly increased the growth parameters and stress resistance of the resulting plants as compared to plants from non-treated seeds, grown under the same conditions.

Thus, the present disclosure provides, in accordance with its first aspect, a method comprising exposing seeds before germination to an aqueous composition comprising at least one NOSH compound containing, covalently bound via a linker moiety, an NO releasing moiety and an H₂S-releasing moiety. The linker is preferably and aspirin or aspirin derivative or a carboxylic acid ester group.

When referring to seeds before germination it is to be understood as seeds that do not exhibit any visible or otherwise measurable sprouting of a seedling through the seed casing. Seeds before germination are referred to herein, at times, as pre-germinated seeds or by the term “seed level”.

The method thus involves treatment of seeds at the seed level. The seeds are treated by exposure thereof to a solution comprising a NOSH compound (a compound containing at least a NO-releasing moiety and an H₂S-releasing moiety, the two moieties being covalently bound via a linker moiety).

In the context of the present disclosure when referring to NO-releasing moiety it is to be understood as a moiety that acts as a NO donor, i.e. releases under suitable conditions from the NOSH compound a nitric oxide. NO donors are known in the art as well as conditions suitable for the release therefrom of nitric oxides.

In some embodiments, the NO releasing moiety is a moiety selected from the group consisting of —NO, —C(O)—(CH₂)ₙ—ONO₂, —O—(CH)ₙ—ONO₂, —(CH₂)ₙ—ONO₂, —C(O)C(CH₃)₂—SNO, —C(O)—(C H₂)—C(CH₃)₂—SNO, —NH—CH₂—C(CH₃)₂—SNO, —CH₂—C(CH₃)₂—SNO,

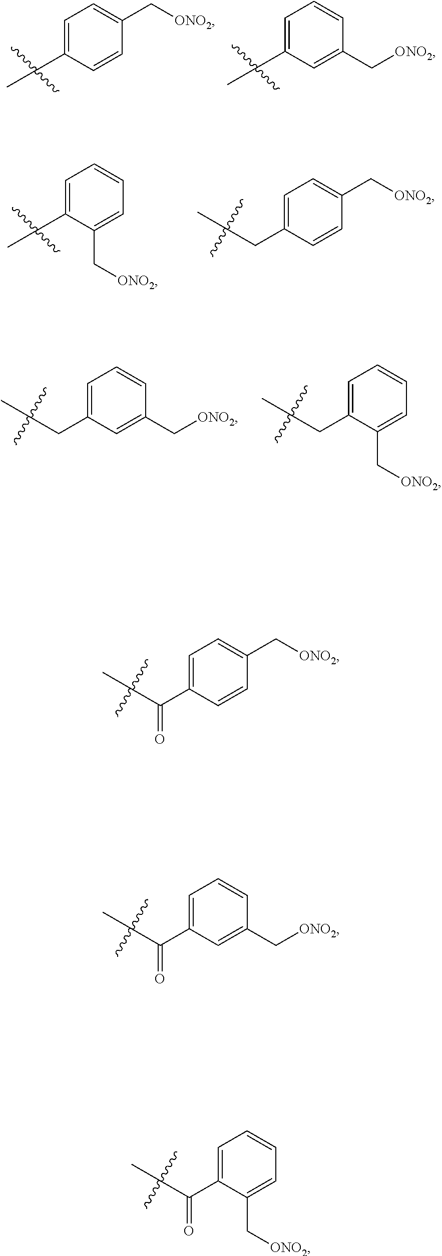

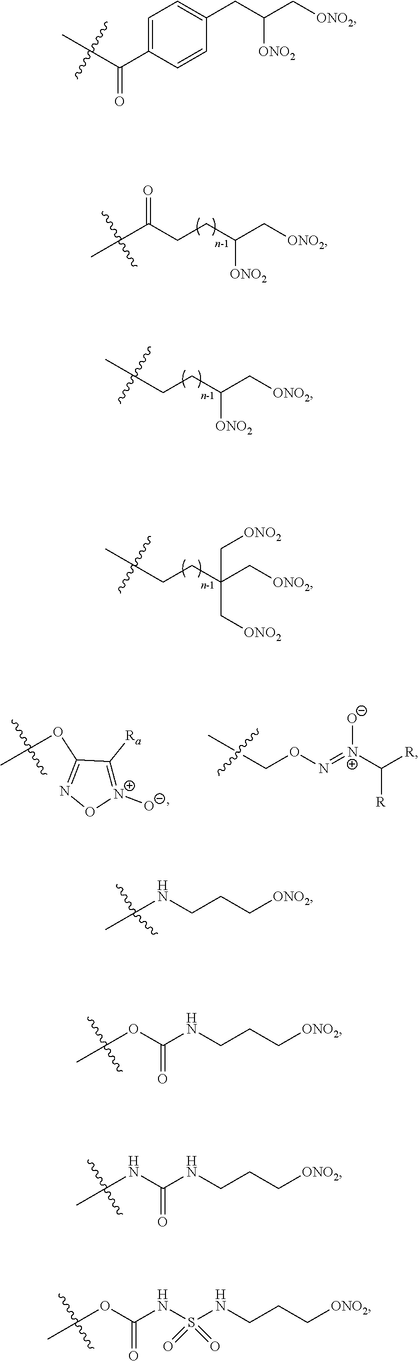

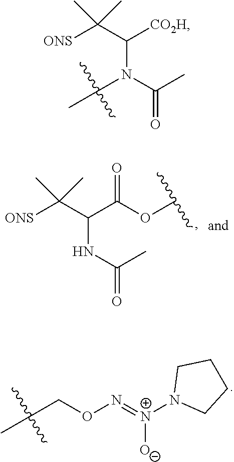

In which n is any integer selected from 1, 2, 3, 4, 5, 6, and 7;

Rₐ is H, C₁-C₁₀ alkyl, aryl, S(O)₂-aryl, CN, or CON(R_{b})₂; and each R_{b}, independently, is s H or C₁-C₁₀ alkyl.

In the context of the above definition, alkyl refers to a saturated, linear or branched hydrocarbon moiety; aryl refers to a hydrocarbon moiety having one or two or more fused aromatic rings. Examples of aryl moieties include phenyl (Ph), naphthyl, pyrenyl, anthryl, and phenanthryl.

Alkyl and aryl mentioned herein include both substituted or unsubstituted moieties, unless specified otherwise. Possible substituents on aryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, C₁-C₁₀ alkylsulfonamino, arylsulfonamino, C₁-C₁₀ alkylimino, arylimino, C₁-C₁₀ alkylsulfonimino, arylsulfonimino, hydroxyl, halo, thio, C₁-C₁₀ alkylthio, arylthio, C₁-C₁₀ alkylsulfonyl, arylsulfonyl, arylsulfonamide, heteroarylsulfonamide, acylamino, aminoacyl, aminothioacyl, amidino, guanidine, ureido, cyano, nitro, nitroso, azido, acyl, thioacyl, acyloxy, carboxyl, and carboxylic ester. On the other hand, possible substituents on alkyl include any of the above-recited substituents except C₁-C₁₀ alkyl.

In the context of the present disclosure, when referring to H₂S-releasing moiety it is to be understood as a moiety that generates from the NOSH compound a hydrogen sulfide (H₂S). H₂S donors are also known in the art as well as conditions suitable for the release therefrom of H₂S.

In some embodiments, H₂S-releasing moiety is selected from the group consisting of

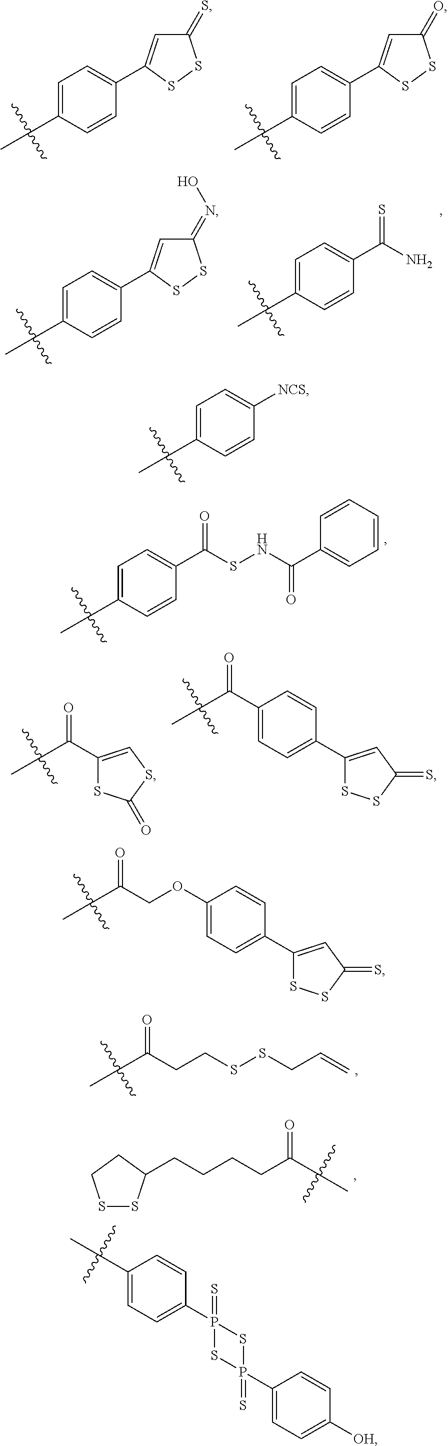

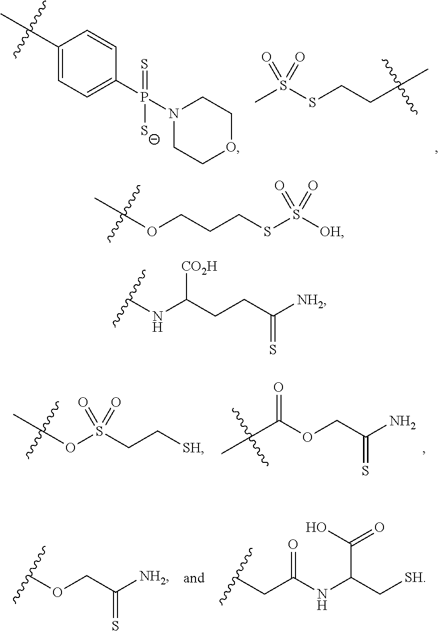

In some embodiments, the linker between the NO-releasing moiety and the H₂S-releasing moiety is a salicylic acid and the NOSH compound has the general formula:

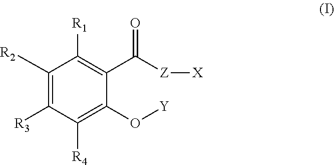

Where Z is O, NH or a valence bond;
X and Y are different, each representing either the NO-releasing moiety or the H₂S-releasing moiety; and
R₁ to R₄ each represent, independently H, halo, C₁-C₁₀ alkyl, or N(R)₂, in which R is H or C₁-C₁₀ alkyl.

In some embodiments, NOSH compounds represented by formula I are known and are referred to herein as NOSH-NSAID, NOSH-aspirin or NOSH-ASA.

In another embodiments, the linkage of the NO-releasing moiety and the H₂S-releasing moiety is via a carboxylic acid ester group —C(O)—O—, in short, ester linkage). Such compounds are encompassed by the term NOSH compound and are referred to as NOSH-esters or NOSH.

A non-limiting list of NOSH compounds is selected from the group consisting of

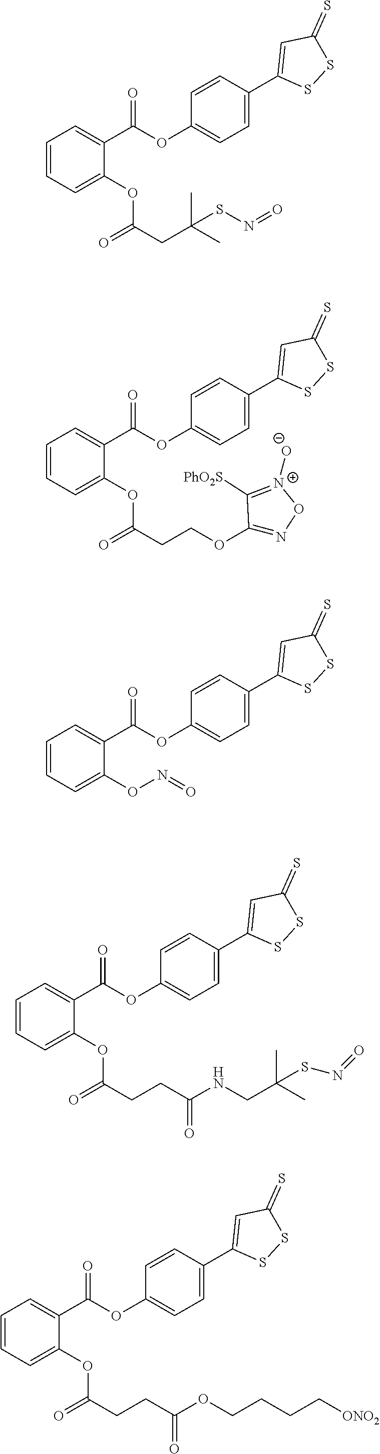

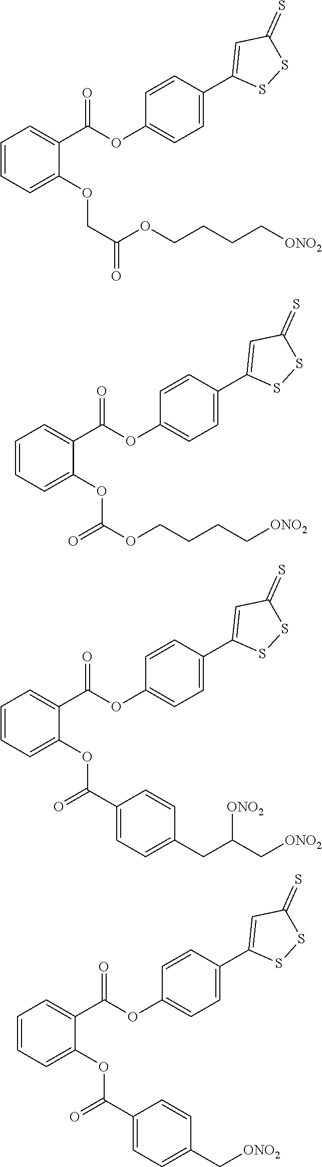

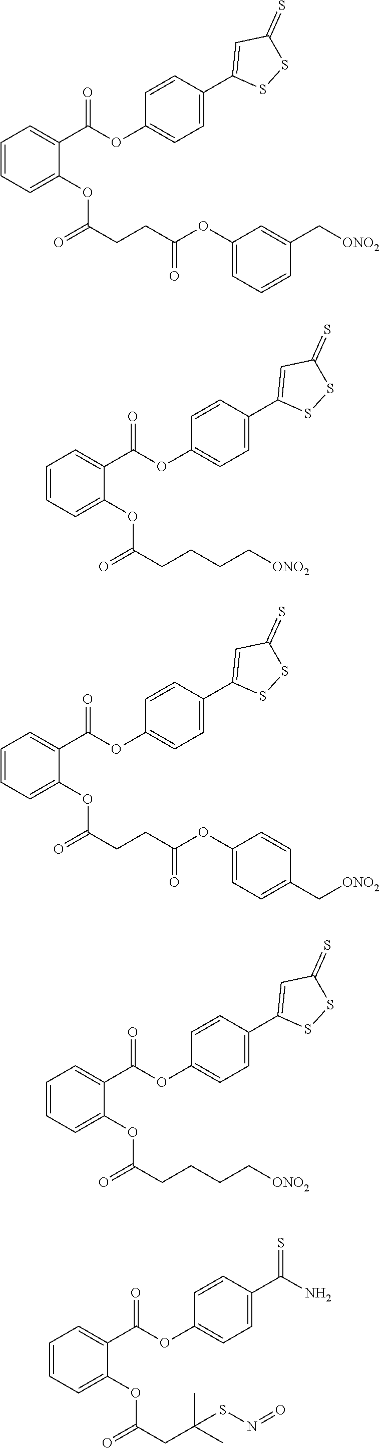

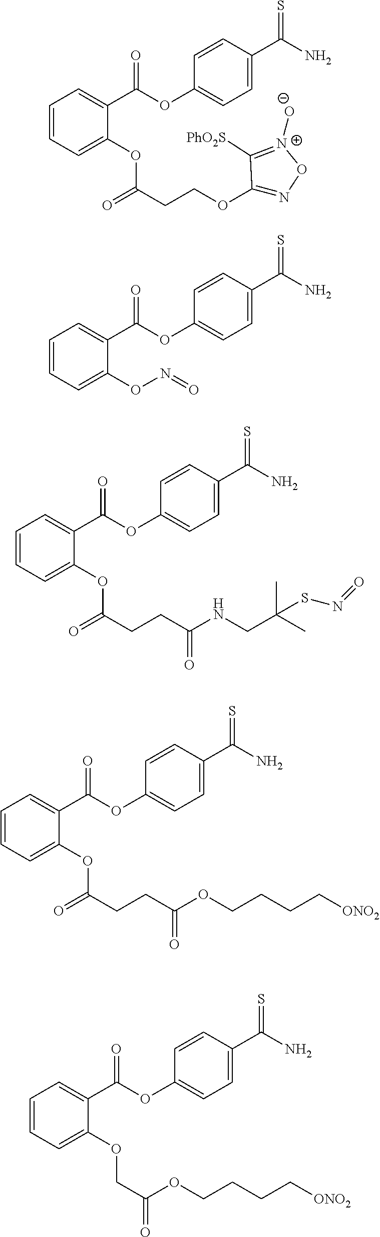

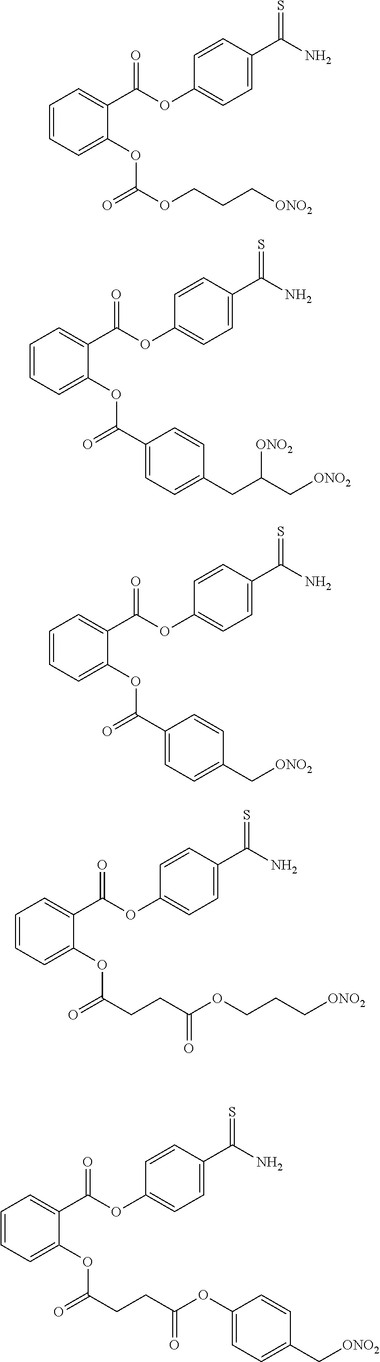

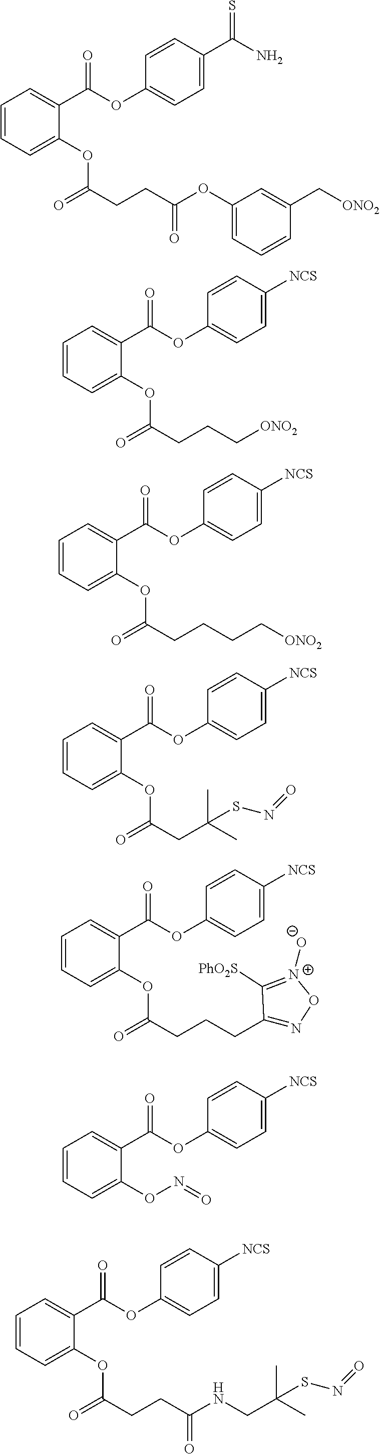

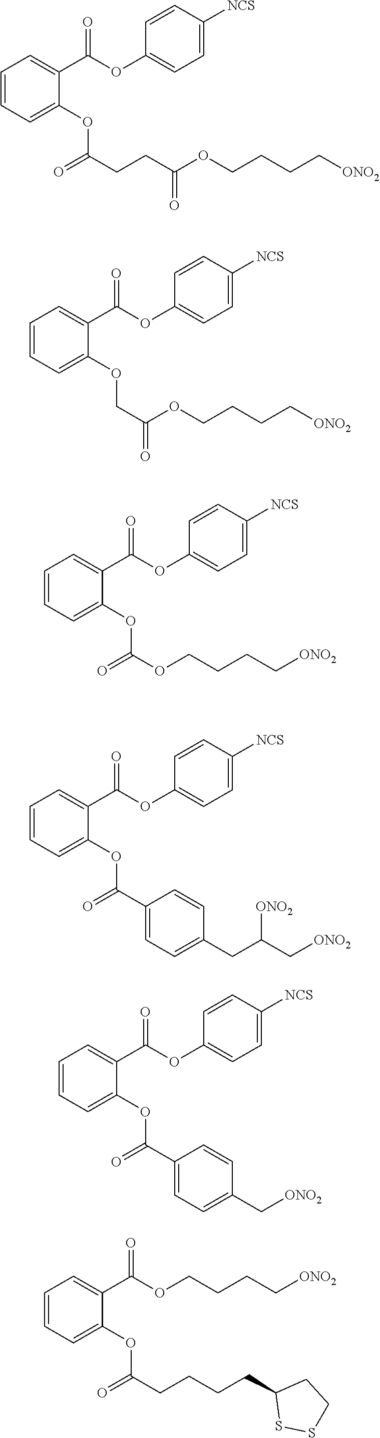

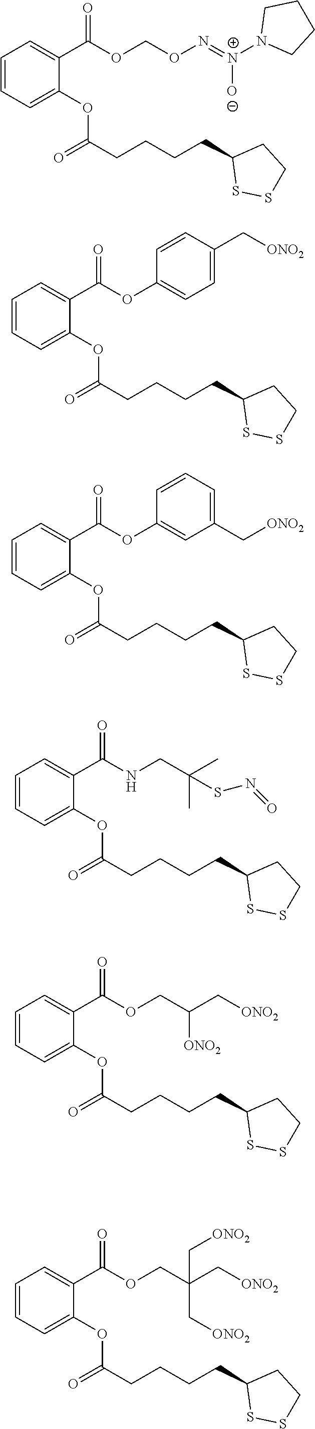

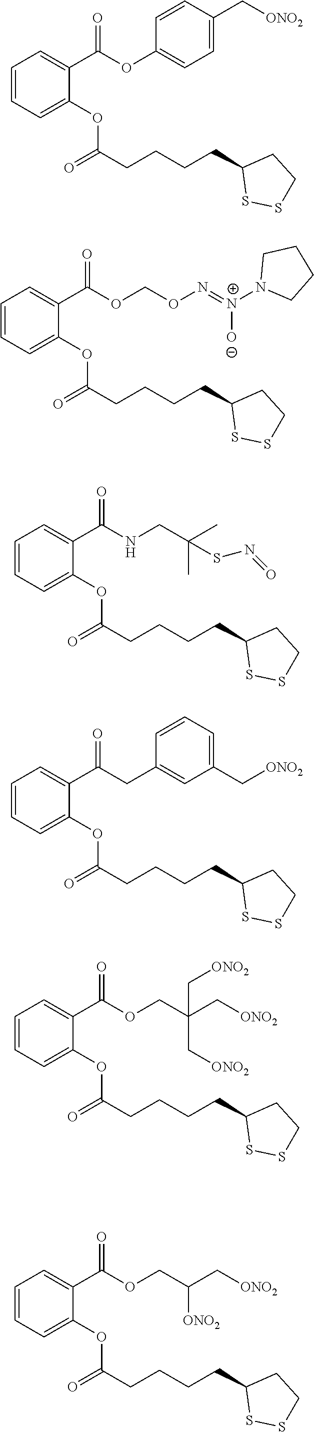

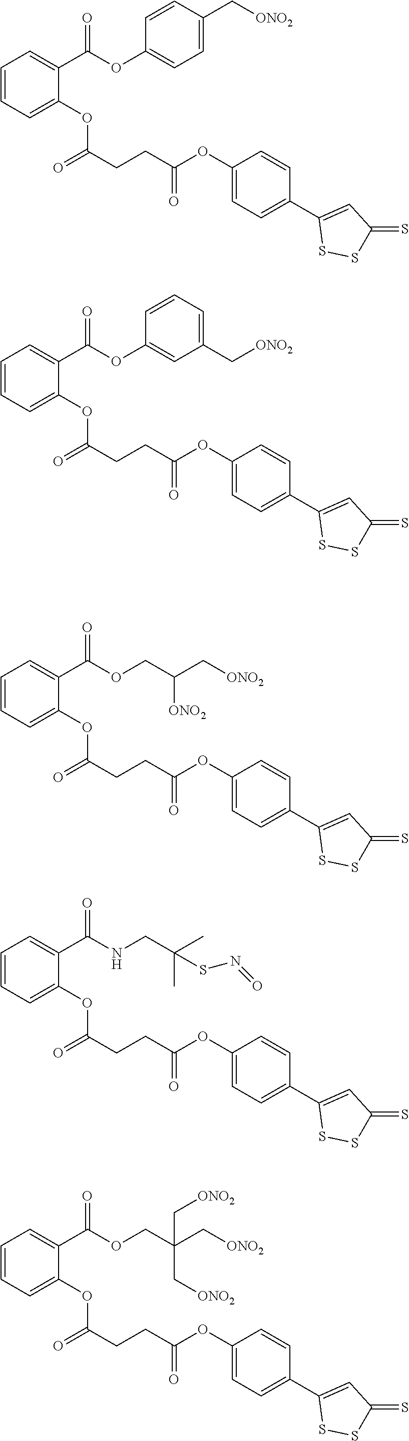

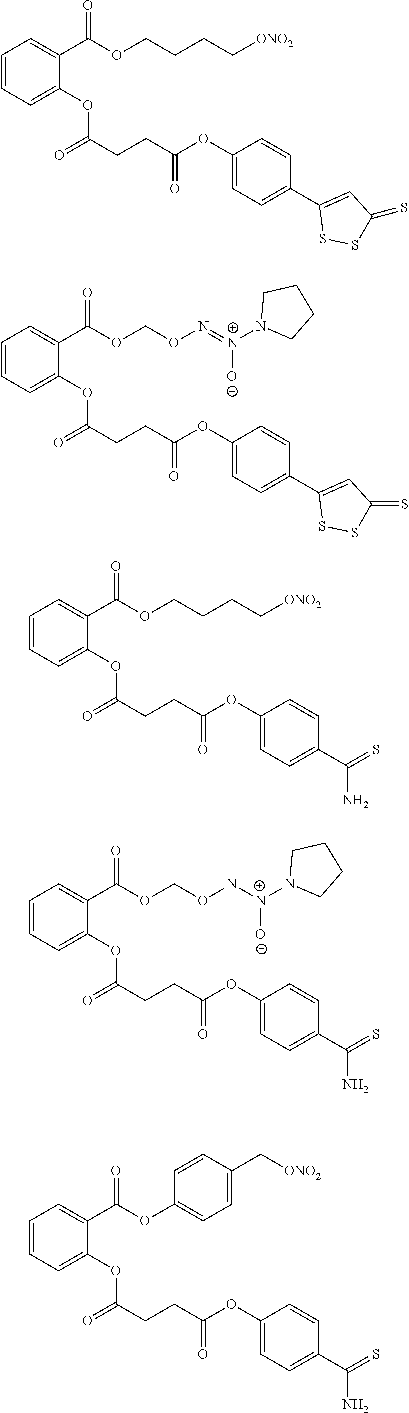

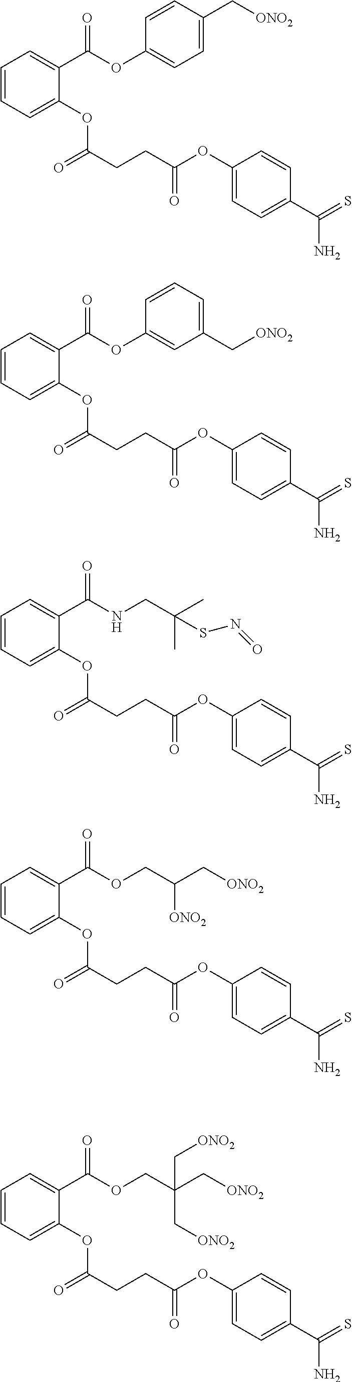

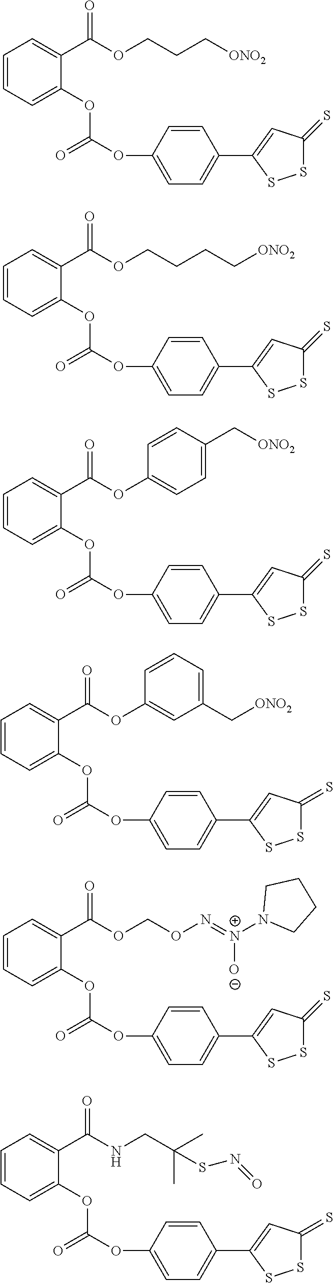

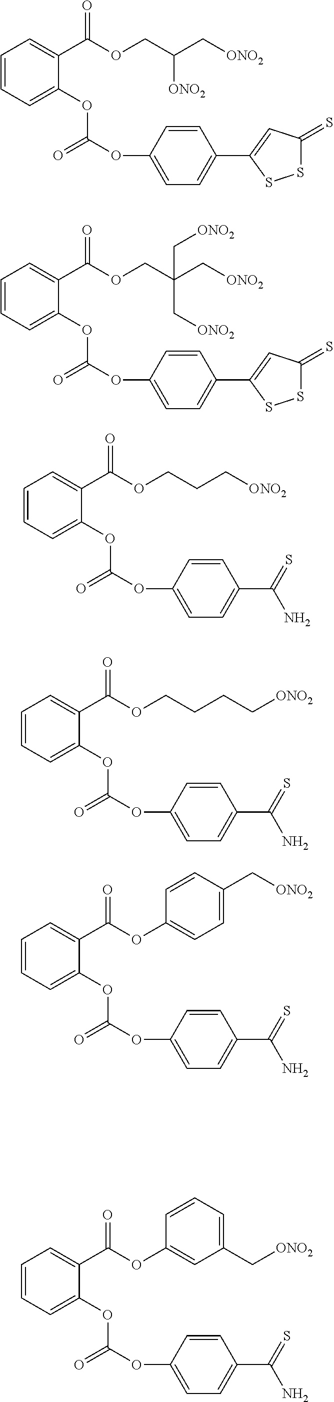

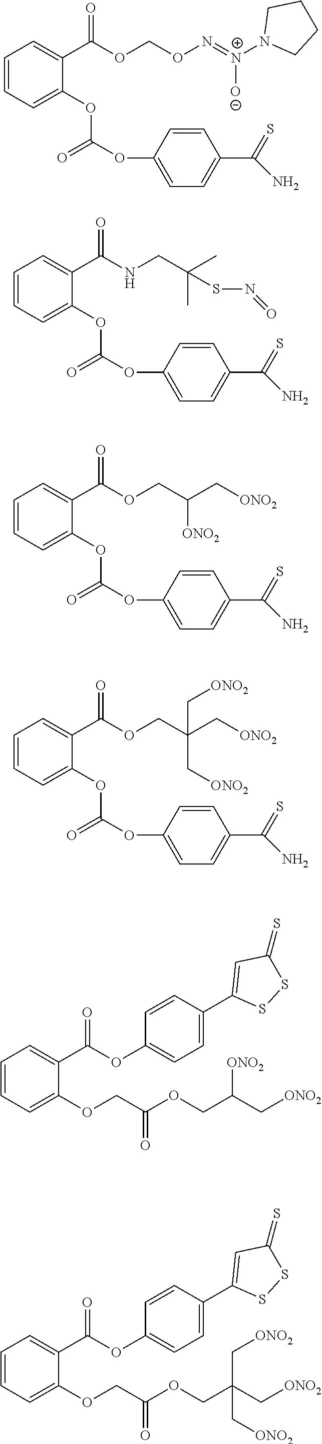

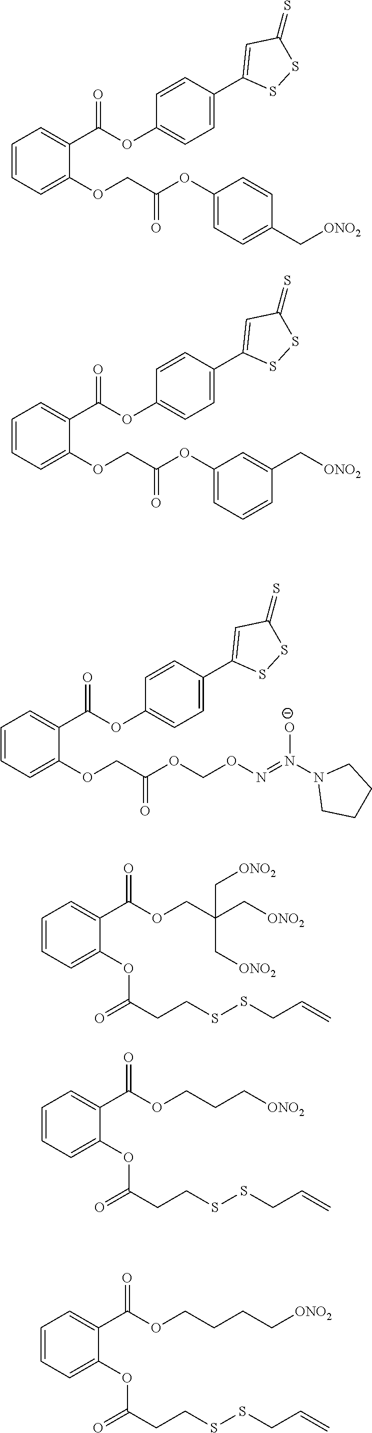

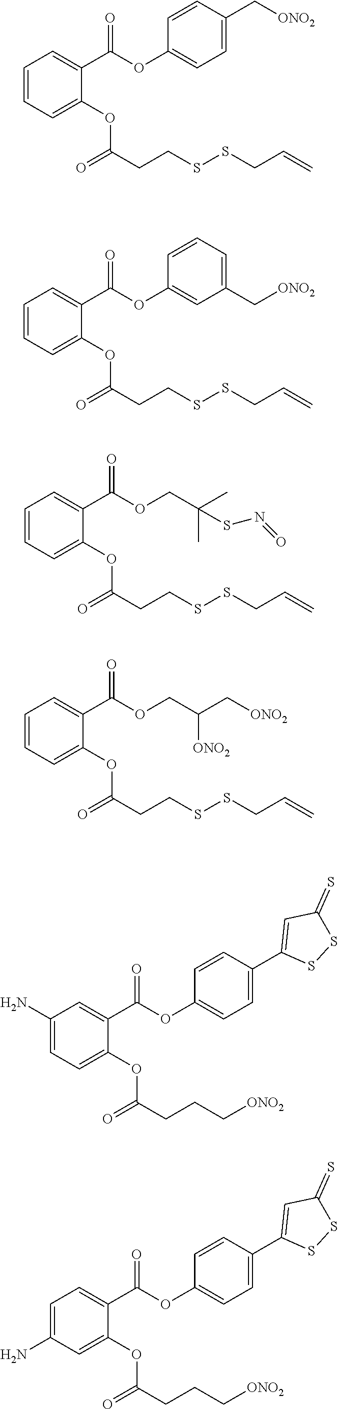

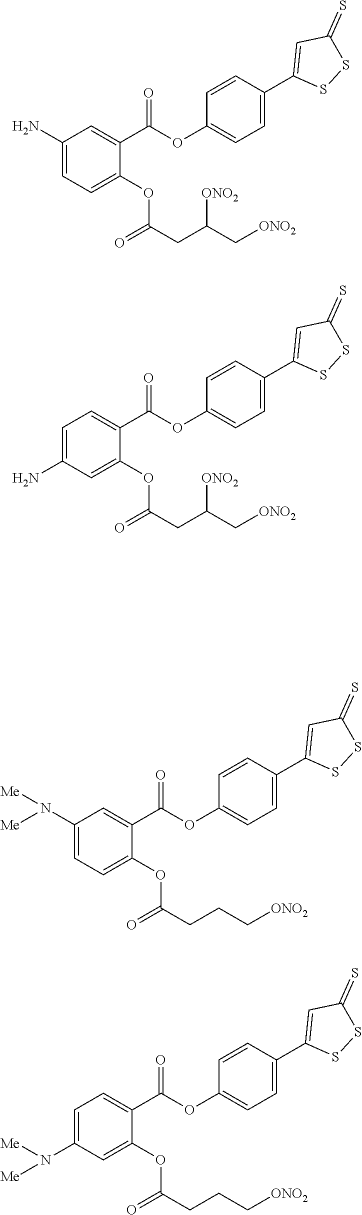

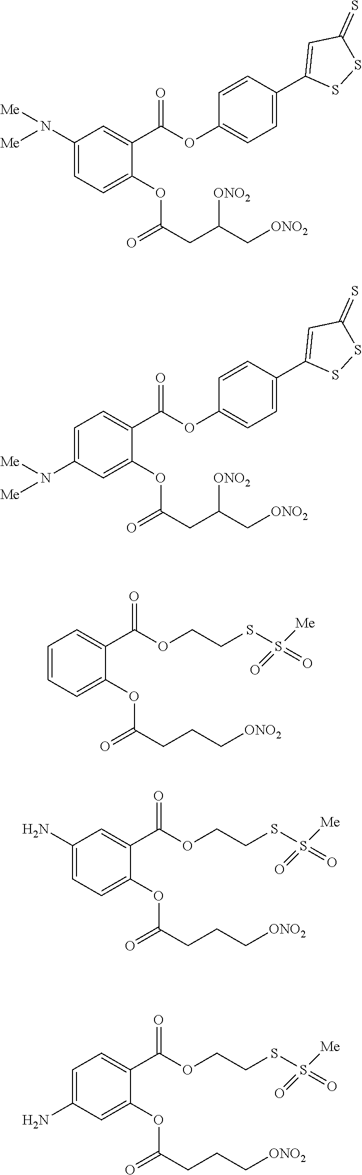

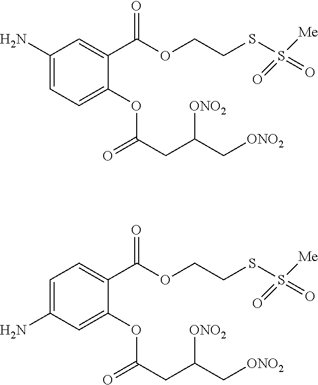

In some embodiments, the H₂S-releasing moiety is selected from

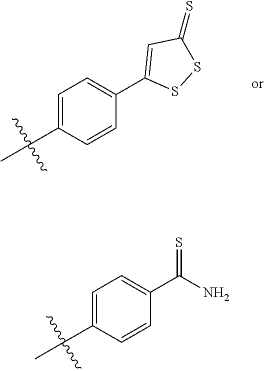 [SMILES: *c1ccc(-c2cc(=S)ss2)cc1 ; *c1ccc(C(N)=S)cc1]

In some embodiments, the NO-releasing moiety is —C(O)—(CH₂)ₘ—ONO₂.

A specific and preferred NOSH compound is one where the NO-releasing moiety and the H₂S-releasing moiety are linked via the ester linkage.

A preferred NOSH compound is one having the formula:

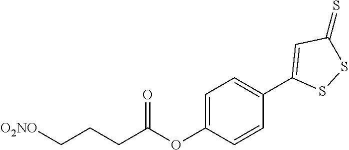

NOSH compounds, either NOSH-NSAID or NOSH-ester are known, the preparation of which is described, inter alia, International Patent Application Publication No. WO 2013/025790, the content of which is incorporated herein, it its entirety, by reference.

The NOSH compound is used according to the present disclosure for treating seeds before germination is initiated and it has been found that such treatment was sufficient for at least priming the seeds for an increased growth and/or for bestowing the plant with protection against stress conditions. This was unexpected in view of previous findings that the treatment requires the seeds to be at least at a germination stage (WO2015/123273). It has now been found that treatment before there is any detected evidence of germination, e.g. where the seeds are maintained intact, is sufficiently beneficial in improving one or more plant parameters, and at least one of said plant growth and/or stress protection. Further, it has been found that there is no need for subsequent treatments of seedlings or the plant in order to maintain such beneficial effect.

In the context of the present disclosure, when referring to treatment of seeds it is to be understood as encompassing any means of applying the NOSH compound (specifically, dissolved in an aqueous composition) directly onto the seeds, when the seeds are at their pre-germination stage.

In the context of the present disclosure, when referring to an aqueous composition it is to be understood as any water containing composition. In some embodiments, the composition contains a mixture of water and an organic solvent, such as any of the polar solvents required for dissolving the NOSH compound (see below), e.g. DMSO.

In the context of the present disclosure, the composition without the NOSH compound is referred herein as the carrier (agriculturally acceptable carrier). In some embodiments, the carrier is 0.1% DMSO.

Treatment can thus include any one or combination of soaking the seeds with the aqueous composition, spraying the seeds with the aqueous composition, applying vacuum to the seeds which are on the aqueous composition (known as vacuum infiltration), dipping the seeds for several times in the aqueous composition or otherwise brining the aqueous composition comprising the NOSH compound into contact with the seeds for a time sufficient to induce the desired effect on the seeds.

In some embodiments, treatment is seed priming and the priming involves at least soaking of the seeds in the aqueous composition comprising the NOSH compound dissolved therein.

In some embodiments, the exposing, and in particular the soaking is for any period sufficient to provide the desired effect (e.g. priming growth, providing stress protection). In some embodiment the exposure is for several seconds, several minutes or for a period of at least 1 hr, at times, between 1 hr and 24 hr, at times, between 1 hr and 20 hr or any time period between 1 minute to 24 hours.

In some embodiments, the soaking takes place in the dark and/or at room temperature (about 25° C.).

The treatment or priming of the seeds results in one or more beneficial effects on the plant parameters. In the context of the present disclosure, when referring to plant parameters it is to be understood as encompassing any one or more of plant growth, plant size, leaf size, plant biomass, root length, root mass, yield (e.g. number of harvest per plant, number of seeds), harvest index (the yield of a crop species versus the total amount of biomass that has been produced. The commercial yield can be grain, tuber or fruit. (http://plantsinaction.science.uq.edu.au/content/641-harvest-index)), crop index (the relative yield of the crops on a particular area with the average yield over an entire region being taken as 100), germination index (GI, GI=Σ (Gi/Ti) where Gi is the germination percentage at the ith day, and Ti is day of germination test. High GI shows high seed quality and performance [Li et al., Plant Growth Regul (2013) 71:31-40]), tolerance to stress conditions (e.g. abiotic or biotic stress tolerance, drought tolerance) etc.

Further, in the context of the present disclosure, the treatment of the seeds provides an increase in the one or more plant parameters, the increase being determined in comparison with the same parameter when measured on a reference plant, the reference plant being the plant arising from the same seeds, when exposed under the same conditions of time, temperature, dark/light to only the carrier (e.g. the solvent diluted in water) and the plant than grown under the same conditions.

In some embodiments, the increase is presented as the numerical ratio between the measured parameter for the treated plant or plant part or plant tissue and the same parameter from a reference value (be it a predetermined reference or one obtained from a carrier treated plant grown alongside the treated seeds). In some embodiments, an increase is concluded when there is a statistically significant difference between the measured parameter obtained from the NOSH treated seeds and the reference plant, this being determined according to acceptable statistical tests. Some statistical tests acceptable in agriculture include, without being limited thereto, one-way analysis of variance (ANOVA), post-hoc analysis, Duncan test, Tukey's test. According to some embodiments, a statistically significant difference is determined with a pvalue that is equal or lower than 0.05, preferably, equal or lower than 0.04, 0.03, 0.02 or even 0.0001.

In yet some embodiments, the increase in a plant parameter is presented by scoring values, e.g. a score of 1 is regarded as the lowest possible value for the selected parameter, and a score of 10 is regarded as the highest possible value for the selected parameter.

In some embodiments, the plant parameter is plant growth. When referring to plant growth it is to be understood as encompassing any measurable value indicative of the plant growth, including weight (fresh or dry, preferably dry) of the plant or weight of plant part (e.g. leaves), projected leaf/rosette area, Relative Growth Rate (RGR), seed production, seed germination index, yield, Leaf Area Index (LAI), Net Assimilation Rate (NAR), Leaf Area Ratio (LAR), Leaf Area Duration (LAD) and Crop Growth Rate (CGR), plant or fruit vigor, fruit color break, fruit firmness, number of fruit per plant, color of leaves.

In some embodiments, the plant parameter comprises the ratio between weight of a plant part, and in some embodiments, of rosettes, from NOSH treated seeds and the same plant part from carrier-treated seeds. The weight can be that of fresh matter or dry matter.

In some embodiments, the plant parameter comprises the ratio between the projected rosette area (PRA) from a plant obtained from NOSH treated seeds and the PRA obtained from carrier-treated seeds.

When the plant parameter is the PRA ratio, a beneficial effect of the seed treatment is obtained or concluded when the ratio is statistically significantly greater than 1 (p equal or lower than 0.05). As shown hereinbelow and without being limited to this specific example, when the seeds were exposed to NOSH-ester containing composition, the PRA has increased in the >1.5 fold range.

In some embodiments, the plant parameter is stress tolerance, i.e. the treatment primes the plant to an increased tolerance to one or more stress conditions. When referring to stress conditions it may be understood as one or combination of drought (unwatering), soil salinity, temperature etc.

The treatment required that the NOSH compounds be prepared by first dissolving the compound with a non-diluted (pure) solvent. Without being bound by theory, it is believed that the use of a non-diluted solvent allows an essentially complete dissolution of the compound, and only then, the solvent is diluted to obtain an aqueous solution of the compound. Typically, although not mandatory, the dilution of the solvent is required due to potential phytotoxicity of the solvent.

The dilution of the solvent carrying the dissolved NOSH compound is to a level where it is no longer phytotoxic. The final concentration of the solvent in the aqueous solution depends on the type of the solvent and the type of the NOSH compound dissolved therein. A person versed in the art can easily determine the upper concentration of the solvent, i.e. the concentration above which the resulting solution turns phytotoxic or otherwise damages the plant cells.

In some embodiments, the concentration of the solvent may range from 0.01% v/v to 30% v/v, at times, from 0.01% v/v to 20% v/v, or to 15% v/v, or to 10% v/v, or to 2.5% v/v, or to 1.5% v/v. In some embodiments, the solvent concentration is between 0.1% v/v to 10% v/v.

The solvent is selected to be a polar solvent.

In some embodiments, the solvent is selected from the group consisting of dichloromethane (DCM), tetrahydrofuran (THF), ethyl acetate, acetonitrile, dimethylformamide (DMF), dimethyl sulfoxide (DMSO), acetone, methanol, ethanol, isoproplyl alcohol, acetic acid and hexamethylphosphoric triamde (HMPT).

In some embodiments, the solvent is selected from the group consisting of dimethylformamide, DMSO, acetone, ethyl acetate, ethyl alcohol.

In some embodiments, the solvent is a polar aprotic solvent.

In some embodiments, the solvent is DMSO.

In some embodiments, the solvent is DMSO, at a concentration of 0.01% v/v to 15% v/v, at times, up to 10%, or up to 9%, 8%, 7%, 6%, 5%, 4%, 3%, 2%, or 1%, yet in some embodiments, at a concentration of 0.1% v/v±0.05% v/v.

The NOSH compound is dissolved in the aqueous solution comprising also the diluted solvent. The concentration of the NOSH compound can vary, and will depend, inter alia, on the type of the NOSH compound and/or the seeds to be treated.

In some embodiments, the concentration of the NOSH compound in the aqueous solution, ready for use on the pre-germinated seed is in the range of 10 nM to 500 μM.

In some embodiments, the concentration of the NOSH compound is between 0.1 μM to 300 μM, at times between 0.1 μM and 200 μM, at times, between 0.1 μM and 150 μM, at times between 5 μM and 100 μM, at times, between 0.1 μM and 80 μM, at times between 5 μM and 70 μM.

In some embodiments, the NOSH compound is one having the salicylic acid linker and the concentration of the NOSH compound is about 10 μM±5 μM.

In some embodiments, the NOSH compound is one having the ester linker and the concentration of the NOSH compound is about 50 μM±5 μM.

The NOSH compound is effective in increasing one or more plant parameters. It has been found that while both NOSH-NSAID and NOSH-ester were effective in at least promoting growth following treatment at the seed level, NOSH-ester was more effective in promoting growth as compared to NOSH-NSAID.

Upon treatment of the seeds, the NOSH compound may be adhered and/or adsorbed by the seeds. Thus, in the context of a further aspect of the present disclosure, also provide are seeds comprising (e.g. adsorbed, adhered or otherwise associated with the seeds) an amount of a NOSH compound, wherein the seeds are at their pre-germinated stage. The presence of the NOSH compound can be detected by any known spectroscopic device, e.g. HPLC.

In some embodiments, the seeds are in dry form. Drying of the seeds can be by placing the soaked seeds in fume hood for 2-3 hours in order to completely dry them.

Finally, in the context of a further aspect of the present disclosure, there is provided a kit or package comprising NOSH compound and a solvent for dissolving the same, or a composition comprising the NOSH compound in dissolved form. The kit also comprises instructions for preparing the composition (when the NOSH compound is provided in non-dissolved form) and for use of the composition for treating seeds at their pre-germination stage.

In some embodiments, the NOSH composition is a stock solution and the instructions also direct on how to dilute the composition with an aqueous medium, e.g. water, to obtain the NOSH composition suitable for treating the seeds (e.g. not phytotoxic concentration).

As used herein, the forms “a”, “an” and “the” include singular as well as plural references unless the context clearly dictates otherwise. For example, the term “a plant” includes one or more plants.

Further, as used herein, the term “comprising” is intended to mean that, for example, the aqueous composition includes the recited compound but not excluding other elements, such as suitable carriers or other components that may be part of the composition for treating the seeds. The term “consisting essentially of” is used to define compositions which include the recited elements but exclude other elements that may have an essential significance on the effect of the composition on the seeds. “Consisting of” shall thus mean excluding more than trace elements of other elements that may have an effect on the seeds. Embodiments defined by each of these transition terms are within the scope of this disclosure.

Further, all numerical values, e.g. when referring the amounts or ranges of the elements constituting the formulation are approximations which are varied (+) or (−) by up to 20%, at times by up to 10% of from the stated values. It is to be understood, even if not always explicitly stated that all numerical designations are preceded by the term “about”.

The invention will now be exemplified in the following description of experiments that were carried out in accordance with the invention. It is to be understood that these examples are intended to be in the nature of illustration rather than of limitation. Obviously, many modifications and variations of these examples are possible in light of the above teaching. It is therefore, to be understood that within the scope of the appended claims, the invention may be practiced otherwise, in a myriad of possible ways, than as specifically described herein below.

### EXAMPLES

### Example 1—Effect of NOSH or NOSH-ASA on Plant Growth

### NOSH or NOSH-ASA Preparation

Stock solutions of NOSH or NOSH-ASA were each prepared by initial dissolution in 100% DMSO. From each stock solution, treatment compositions at a 0-100 μM concentration gradient (diluted in 0.1% v/v DMSO) were prepared.

### Seeds Treatment

Arabidopsis thaliana seeds (ecotype Col-0) were treated at their pre-germination stage with different concentrations of NOSH or NOSH-aspirin solutions (0-100 μM concentration gradient) by soaking the seeds in Petri dishes at room temperature and in the dark. After 12 hrs, the seeds were air dried for 2-3 hrs.

Four seeds per pot were sown in soil medium and stratified at 4° C. for 4 days to achieve germination uniformity. Then the pots were transferred to the growth room and allowed to grow under controlled conditions of 22° C., and a 16-hr day (100-120 μmol m⁻² s⁻¹)/8-hr night regime for 22 days (d). Nine Days After Stratification (DAS), one seedling, which had the projected rosette area closest to the median area of the treatment, was selected from the four seedlings sown per pot. Four independent experiments were carried out using a minimum of 6-8 independent plants as replicates per experiment.

Projected Rosette Area (PRA) was determined by taking pictures of the rosettes 22 days after stratification (DAS). The pictures were subsequently analyzed using ImageJ software (http://rsb.info.nih.gov/ij/) to measure the size of each plant. Represented rosette sizes were calculated by taking the average of the individual plant per repeat and then per treatment.

The same rosettes were used also for fresh weight measurements. In this connection it is noted that in preliminary experiments no substantial differences between the ratio of fresh weight and the ratio of dry weight under optimum soil water content was observed.

For the leaf area analysis, 3^{rd} true leaves were harvested from all the replicates and then the leaves were cleared in 100% ethanol, mounted in lactic acid on microscope slides, and photographed. Leaf areas were measured with the ImageJ software (http://rsb.info.nih.gov//ij/). Abaxial epidermal cells were drawn with a DMLB microscope (Leica) fitted with a drawing tube and a differential interference contrast objective. Drawings were scanned and analyzed using automated image analysis algorithms (Andriankaja et al. 2012).

Subsequently, drawings were used to measure average cell size, from which the total pavement cell number was calculated. The stomatal index was defined as the percentage of stomata in relation to the number of epidermal cells.

Statistical analysis was performed with SAS (Version 9.4 of the SAS System for windows 7 64 bit by using the mixed model analysis. Copyright C 2002-2012 SAS Institute Inc. Cary, NC, USA (www.sas.com).

### Determination of Optimal Compound Concentration

In order to select the optimum concentration of NOSH and NOSH-ASA, a concentration gradient was performed. To achieve the gradient, a stock solution of 100 mM was prepared. At this concentration the compounds did not completely dissolve directly in 0.1% DMSO (visible observation). Therefore, it was decided to prepare a stock solution using 100% DMSO and then dilute the composition to 0.1% DMSO.

The selection of DMSO as the solvent was based on previous studies showing that dissolving those compounds is best achieved with DMSO, [Chattopadhyay et al., 2012]. In order to minimize the solvent effect on the seeds, the percentage thereof was scaled down.

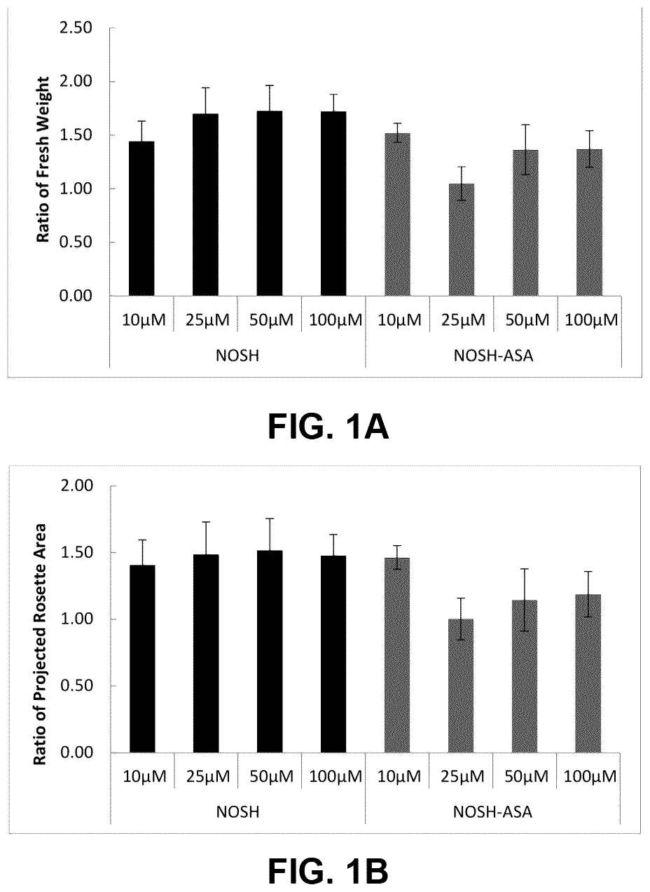

Initial screening at the concentration gradient of 0-100 μM of NOSH or NOSH-ASA demonstrated that seed treatment (at their pre-germination stage) with NOSH applied at 50 μMor with NOSH-ASA applied at 10 μM resulted in highest increase in fresh weight according to the results presented in FIGS. 1A-1B.

Specifically, FIG. 1A is a bar graph showing the ratio of fresh weight of rosettes (22 days after stratification, DAS) received from seeds treated (at pre-germinated stage) and the weight of rosettes at same stage, received from carrier-treated seeds (seeds treated with 0.1% DMSO and without the NOSH compound, (control plants)). The shown values are averages of four to six biological repeats, with each repeat consisting of 6-8 plants.

FIG. 1B is a bar graph showing the ratio of projected rosette area of plants treated at seed level (pre-germination stage) with NOSH or NOSH-aspirin compared with plants from carrier-treated seeds (control plants). The shown values are averages of four to six biological repeats, with each repeat consisting of 6-8 plants.

As noted above, these results determined the optimal concentration on growth parameters for NOSH or NOSH-ASA.

In view of the above results, in the following experiments, NOSH was applied to pre-germinated seeds at a concentration of 50 μM and NOSH-ASA was applied to pre-germinated seeds at a concentration of 10 μM.

### Determination of Effect of Treatment of Pre-Germinated Seed

Seeds were treated by seed soaking for 12 hr with NOSH at 50 μM. Four seeds were then sown in soil medium within a pot and allowed to grow for 22 days under the conditions described above.

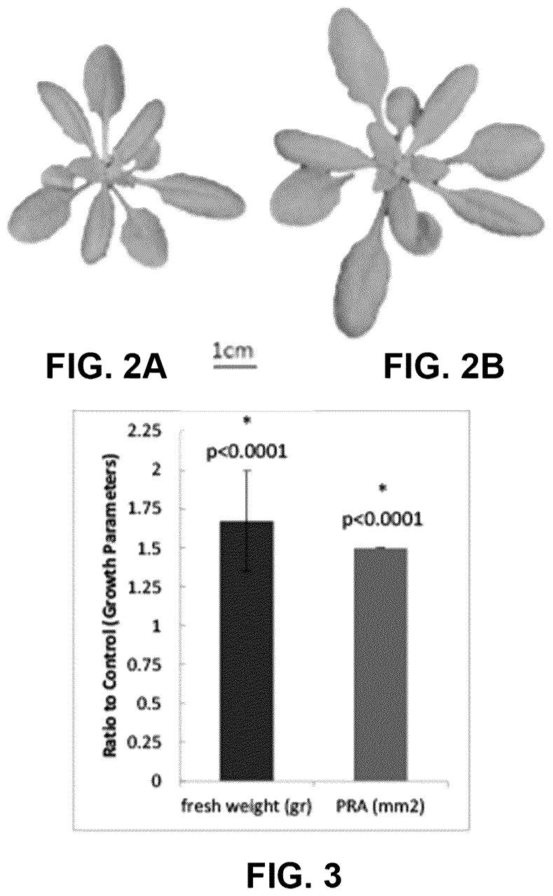

FIGS. 2A-2B are images of representative phenotype of Arabidopsis plants 22 DAS without seed treatment with NOSH (FIG. 2A) or following treatment by soaking the seeds for 12 hr with 50 μM NOSH as described (FIG. 2B).

It is evident that the soaking of the seeds prior to germination with NOSH increased growth of the leaves.

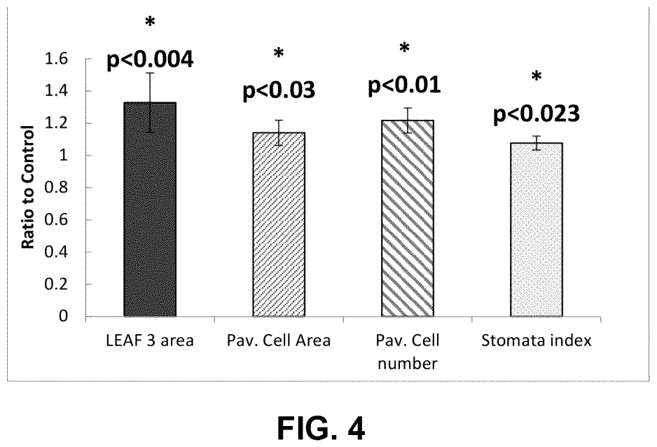

In addition, the ratio of fresh weight of rosettes and PRA of plants received following soaking of the seeds as described were determined. The results are presented in FIG. 3 (values are averages of three biological repeats, with each repeat consisting of 6-8 plants. The results are statistically significant, with a p value of 0.0001.

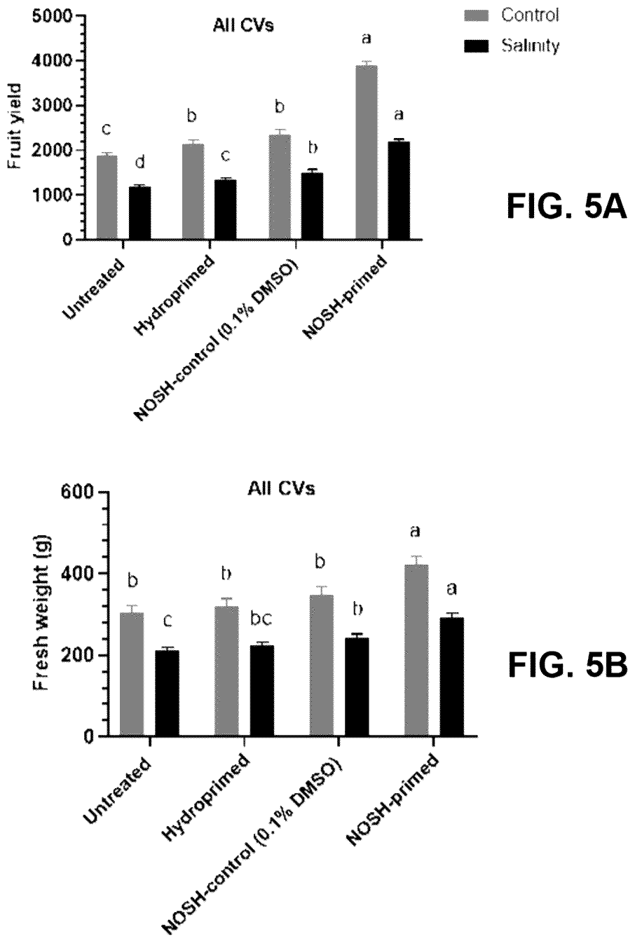

Finally, the ratio of leaf 3 area, pavement cell area, pavement cell number and stomata index measured on plants treated at seed level with 50 μM NOSH compared with plants received from carrier-treated seeds (seeds treated with 0.1% DMSO) were determined, the results of which is provided in FIG. 4 (values are averages of three biological repeats, with each repeat consisting of 6-8 plants).

The results demonstrate a statistically significant (p values indicated in the Figures) increase in all measured parameters (fresh weight, projected rosette area, leaf 3 area, pavement cell area, pavement cell number and stomata index).

### Example 2—Stress Protection of NOSH or NOSH-ASA

Seeds were treated with a NOSH composition comprising 30% methanol and the priming application method was vacuum infiltration (90 sec). Because of the high percentage of methanol solvent, there was no seed germination when soaking was applied for 4 h, 12 h and 24 h. Plants were growth on optimum condition until 37 days and then were left un-watered for 7 days. Observation on plant phenotype was performed and interesting differences on the plant appearance were recorded. For example, the NOSH-treated drought plants were greener and with less anthocyanin on the leaves compared with carrier-treated drought plants. This supported the conclusion that NOSH compounds enhance plant tolerance, (probably, however, without being bound to this hypothesis, by avoiding the accumulation of stress-related pigments such as anthocyanin).

### Example 3—Additional Effects of NOSH or NOSH-ASA

In a similar manner, the effect on growth parameters and stress tolerance upon exposure to drought and/or salinity conditions is examined. Specifically, physiological, biochemical and molecular parameters (porometry, fluorometry, reactive species content, NO biosynthesis and antioxidant enzyme activity assays, RT-qPCR of major cell cycle and defense-related transcripts; for example, see Christou et al., 2013; Filippou et al., 2016) are measured.

### Example 4—A Method of Treating Seeds and Seeds Produced Thereby

A experiment has been carried out, whereby the effect of NOSH-A pre-treatment of tomato seeds was examined under salt stress conditions. The treatments were as follows:
Untreated tomato seeds (4 different varieties—CV1-4)
Hydroprimed seeds (soaked in dH₂O)
NOSH-A control treated seeds (soaked in 0.1% DMSO)
NOSH-A primed seeds

### Plant Material, Stress Conditions and Treatments

NOSH-A was initially prepared in a stock solution of 50 mM using DMSO. The working solution was made by diluting the stock in water to reach a final concentration of 50 μM in 0.1% DMSO. Seed priming was performed by soaking the seeds in NOSH-A solution for 12 hours at 25° C. Then the seeds were placed in gauze under the laminar flow for air-drying, till reaching their initial weight. The seeds were sown in plastic seedling trays (1 seed per pot) filled with sterile soil, covered with a transparent film, and let to germinate in a growth chamber room under certain conditions of 24/20° C. day/night temperatures, 60-70% RH, with a photosynthetic photon flux density of 120 μmol m² s⁻¹ and a 16/8-h photoperiod. At day 7 after sowing, seedlings were transplanted into square plastic pots filled with sterilized pot soil, transferred into a glasshouse, and let to grow until day 24. Growing plants were watered three times per week. Plants were then watered with saline solution (7.8-12.0 dS/m), while controls were watered with dH₂O, until fruiting. A total of four harvests were carried out, with yield data being the total of the four harvesting events. All analyses were carried out using six independent plants per treatment.

### Results and Discussion

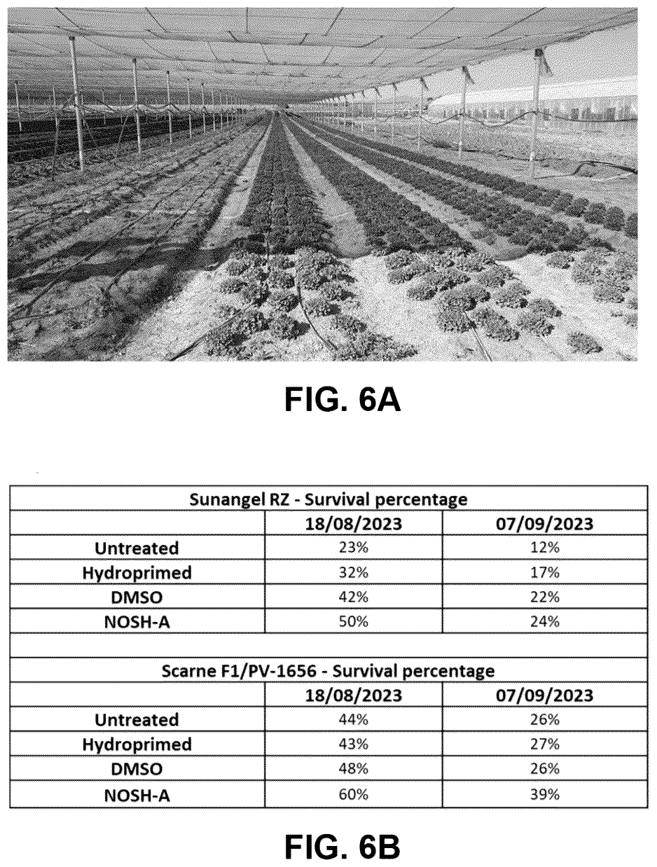

Fruit yield per plant revealed significant improvement following NOSH-A seed priming under both control and high salinity conditions (FIG. 5a), revealing the superior performance of the priming agent both compared with untreated seeds, hydroprimed seeds as well as solvent-treated seeds. Similar significant improvements were also observed in fruit fresh weight (FIG. 5b) and dry weight (FIG. 5c).

### Example 5—A Method of Priming Seeds Growing Under Heat Stress

### Plant Material, Stress Conditions and Treatments

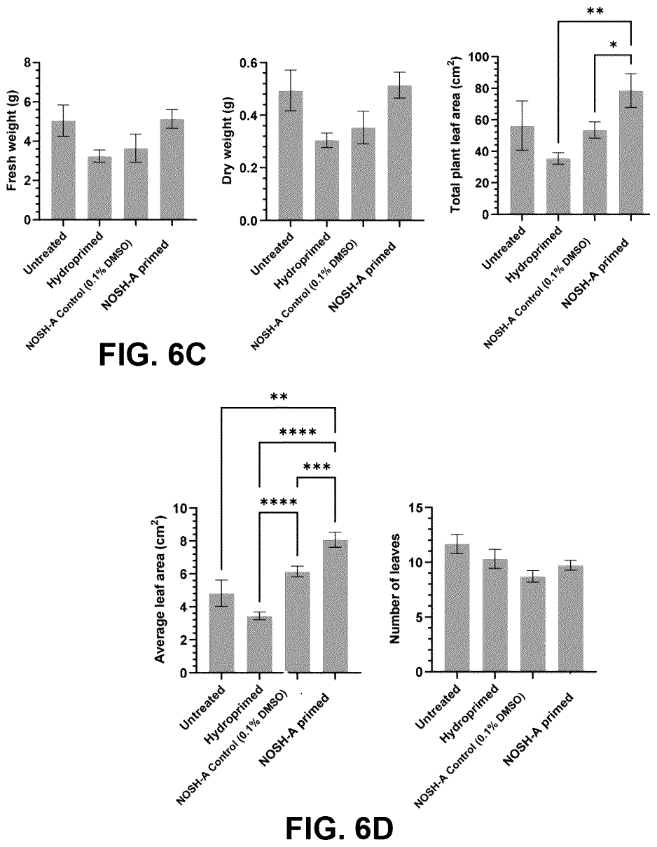

NOSH-A was initially prepared in a stock solution of 50 mM using DMSO. The working solution was made by diluting the stock in water to reach a final concentration of 50 μM in 0.1% DMSO. Seed priming was performed by soaking the seeds of two baby spinach cultivars (Sunangel RZ, Scarne F1PV-1656) in NOSH-A solution for 12 hours at 25° C. Then the seeds were placed in gauze under the laminar flow for air-drying, till reaching their initial weight. Seeds were sown in the soil in a commercial greenhouse (FIG. 6a), and let to grow until day 30. Average day temperature ranged between 37-40° C., while night temperature ranged between 21-23° C. Growing plants were watered three times per week. All agronomic analyses were carried out using a minimum of nine independent plants per treatment, while survival percentage was calculated from a total of 300 plants per treatment.

### Results and Discussion

Survival percentage of plants revealed clear improvement following NOSH-A seed priming (FIG. 6b), revealing the superior performance of the priming agent compared with untreated seeds, hydroprimed seeds as well as solvent-treated seeds in a genotype-independent manner. Similar significant improvements were also observed in total plant leaf area (FIG. 6c) and average leaf area (FIG. 6d).

1. A method of treating seeds to promote plant growth from the seeds comprising exposing seeds to an aqueous composition comprising a compound containing an NO-releasing moiety and an H₂S-releasing moiety covalently bonded together, a compound containing an NO-releasing moiety and an H₂S-releasing moiety covalently bonded to an aspirin derived core, or combinations thereof; and a polar solvent, when the seeds are pre-germination.

2. A method of treating seeds to induce tolerance to stress conditions comprising exposing seeds to an aqueous composition comprising a compound containing an NO-releasing moiety and an H₂S-releasing moiety covalently bonded together, a compound containing an NO-releasing moiety and an H₂S-releasing moiety covalently bonded to an aspirin derived core, or combinations thereof; and a polar solvent, when the seeds are pre-germination.

3. The method of claim 1, wherein the compound containing an NO-releasing moiety and an H₂S-releasing moiety covalently bonded together is of formula II:

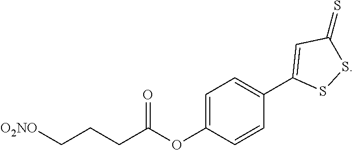

4. The method of claim 1, wherein the compound containing an NO-releasing moiety and an H₂S-releasing moiety covalently bonded to an aspirin derived core is of formula I: 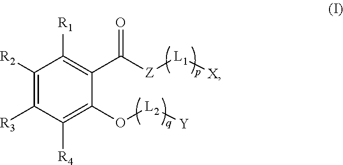
wherein:
each of p and q, independently, is 0 or 1;
each of L₁ and L₂, independently, is a linker, the linker being —C(O)—, —(CH₂)ₘ—, —(CH₂)ₘ—O—, —(CH₂)ₘ—C(O)—, —(CH₂)ₘ—C(O)O—, —(CH₂)ₘ—OC(O)O—, —C(O)—(CH₂)ₘ—O—, —C(O)—(CH₂)ₘ—C(O)—, —OC(O)—(CH₂)ₘ—O—, —OC(O)—(CH₂)ₘ—C(O)—, or —OC(O)—(CH₂)ₘ—C(O)O—, in which m is 1, 2, 3, 4, 5, 6, or 7;
X is a H₂S-releasing moiety or a NO-releasing moiety;
Y is a NO-releasing moiety or a H₂S-releasing moiety, provided that X and Y are not simultaneously H₂S-releasing moieties or NO-releasing moieties;
Z is O or NH; and
each of R₁, R₂, R₃, and R₄, independently, is H, halo, C₁-C₁₀ alkyl, or N(R)₂, in which R is H or C₁-C₁₀ alkyl,
wherein
the H₂S-releasing moiety is 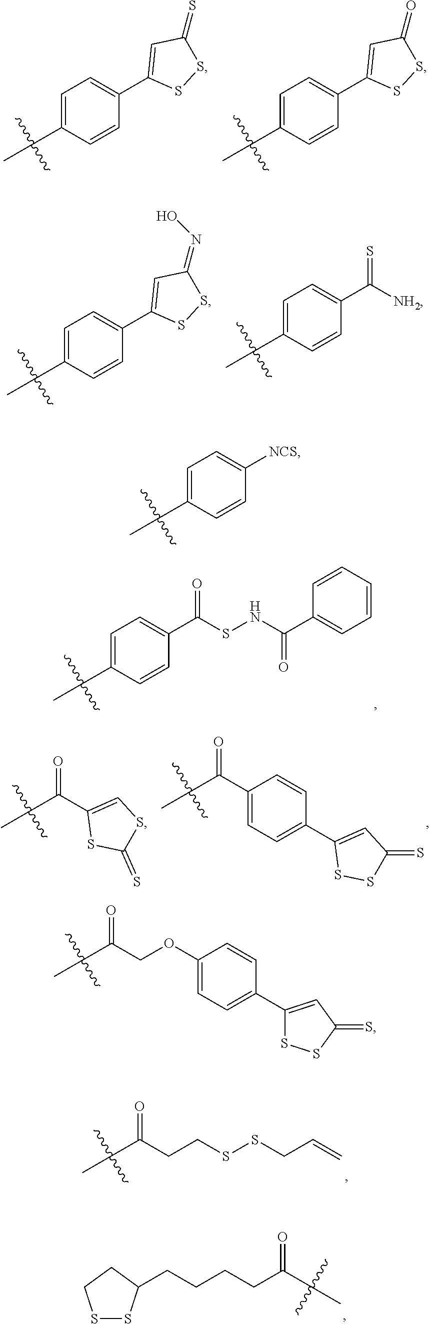 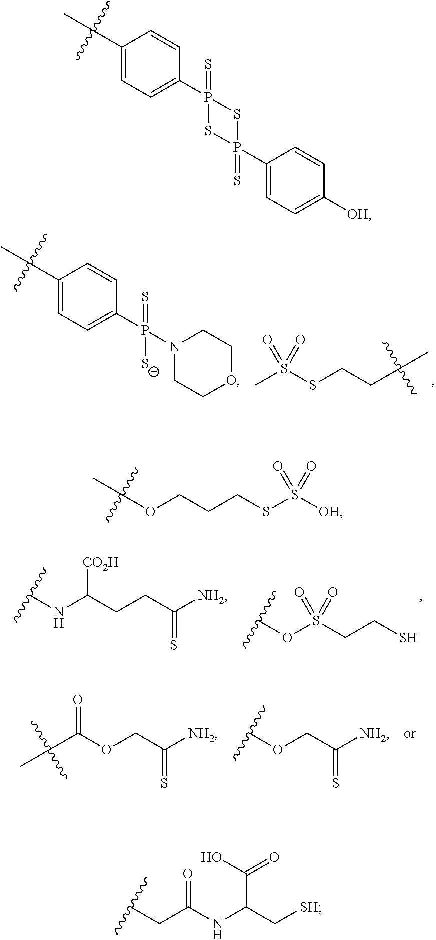
and
the NO-releasing moiety is —NO, —C(O)—(CH₂)ₙ—ONO₂, —O—(CH₂)ₙ—ONO₂—(CH₂)—ONO₂, —C(O)—CH₂—C(CH₃)₂—SNO, —NH—CH₂—C(CH₃)₂—SNO, —CH₂—C(CH₃)₂—SNO, 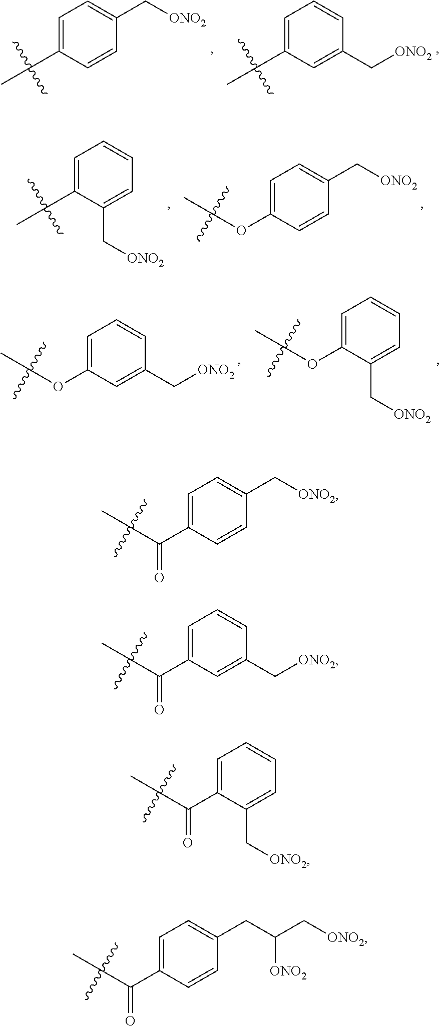 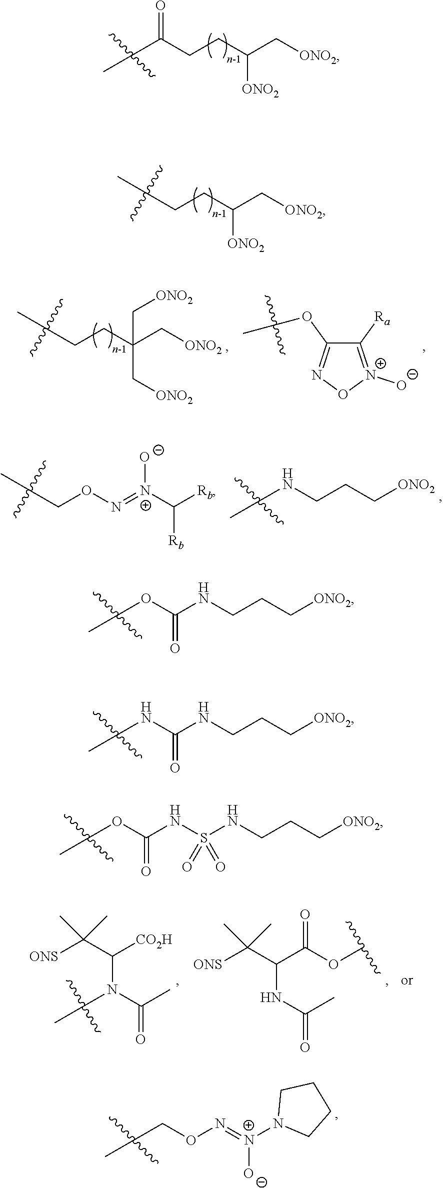
in which n is 1, 2, 3, 4, 5, 6, or 7;
Rₐ is H, C₁-C₁₀ alkyl, aryl, S(O)₂-aryl, CN, or CON(R_{b})₂; and each R_{b}, independently, is H or C₁-C₁₀ alkyl.

5. The method of claim 1, wherein exposing the seeds comprises soaking the seeds in the aqueous composition.

6. The method of claim 1, wherein the compound or compounds are present in a concentration of up to about 100 μM in the aqueous composition.

7. The method of claim 1, wherein the compound or compounds are present in a concentration of from about 1 μM to about 100 μM.

8. The method of claim 1, wherein the compound or compounds are present in a concentration of from about 10 μM to about 50 μM.

9. The method of claim 1, wherein the polar solvent is selected from the group consisting of dichloromethane (DCM), tetrahydrofuran (THF), ethyl acetate, acetonitrile, dimethylformamide (DMF), dimethyl sulfoxide (DMSO), acetone, methanol, ethanol, isoproplyl alcohol, acetic acid, and hexamethylphosphoric triamde (HMPT).

10. The method of claim 1, wherein the polar solvent is a polar aprotic solvent.

11. The method of claim 1, wherein the polar solvent is DMSO.

12. The method of claim 1, wherein the polar solvent is in a concentration of about 0.01% v/v to about 15% v/v.

13. The method of claim 1, wherein the aqueous composition comprises about 0.1% v/v±0.05% v/v DMSO.

14. The method of claim 1, comprising one or more treatments of the seeds prior to germination.

15. The method of claim 14, wherein the treatment is devoid of subsequent exposure of the seeds after germination or a plant grown from said seeds to a NOSH compound.

16. The method of claim 1, wherein the aqueous composition is made by
a. dissolving the compound or compounds in an analytical grade polar solvent; and
b. diluting the dissolved compound or compounds with water to obtain an aqueous composition comprising the dissolved compound or compounds.

17. Pre-germinated seeds comprising a compound containing an NO-releasing moiety and an H₂S-releasing moiety covalently bonded together, a compound containing an NO-releasing moiety and an H₂S-releasing moiety covalently bonded to an aspirin derived core, or combinations thereof.

18. The pre-germinated seeds of claim 17, wherein the compound containing an NO-releasing moiety and an H₂S-releasing moiety covalently bonded together is of formula II: 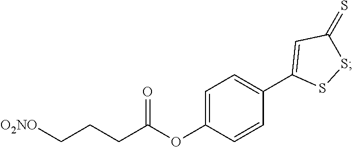
and
the compound containing an NO-releasing moiety and an H₂S-releasing moiety covalently bonded to an aspirin derived core is of formula I: 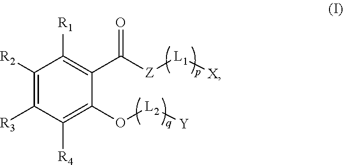
wherein:
each of p and q, independently, is 0 or 1;
each of L₁ and L₂, independently, is a linker, the linker being —C(O)—, —(CH₂)ₘ—, —(CH₂)ₘ—O—, —(CH₂)ₘ—C(O)—, —(CH₂)ₘ—C(O)O—, —(CH₂)ₘ—OC(O)O—, —C(O)ₘ—CH₂)—O—, —C(O)—(CH₂)ₘ—C(O)—, —OC(O)—(CH₂)ₘ—O—, —OC(O)—(CH₂)ₘ—C(O)—, or —OC(O)—(CH₂)ₘ—C(O)O—, in which m is 1, 2, 3, 4, 5, 6, or 7;
X is a H₂S-releasing moiety or a NO-releasing moiety;
Y is a NO-releasing moiety or a H₂S-releasing moiety, provided that X and Y are not simultaneously H₂S-releasing moieties or NO-releasing moieties;
Z is O or NH; and
each of R₁, R₂, R₃, and R₄, independently, is H, halo, C₁-C₁₀ alkyl, or N(R)₂, in which R is H or C₁-C₁₀ alkyl,
wherein
the H₂S-releasing moiety is 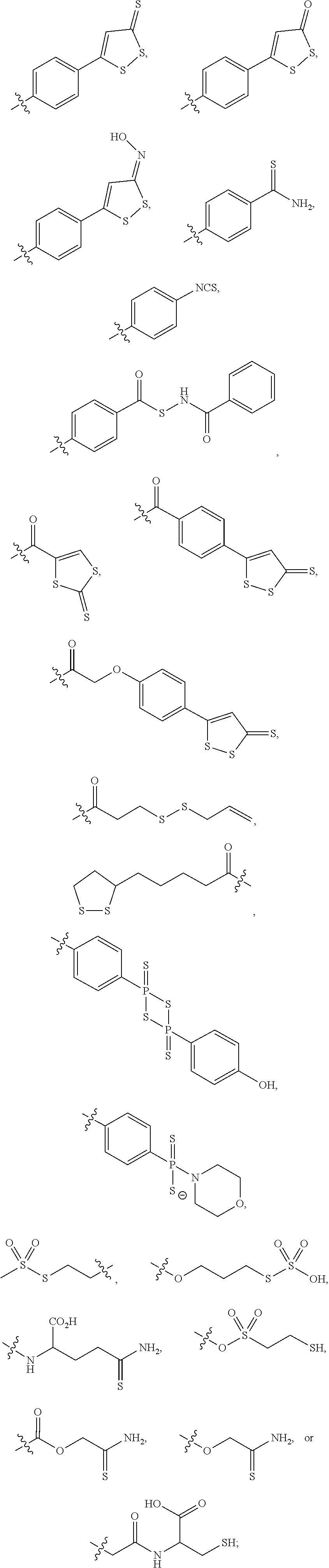
and
the NO-releasing moiety is —NO, —C(O)—(CH₂)ₙ—ONO₂, —O—(CH₂)ₙ—ONO₂, —(CH₂)ₙ—ONO₂, —C(O)—CH₂—C(CH₃)₂—SNO, —NH—CH₂—C(CH₃)₂—SNO, —CH₂—C(CH₃)₂—SNO, 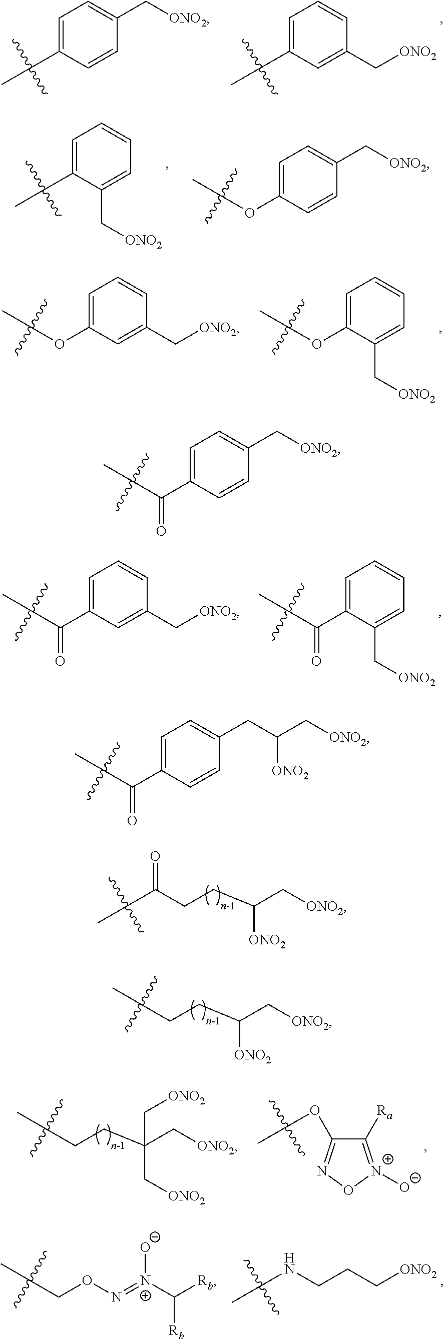 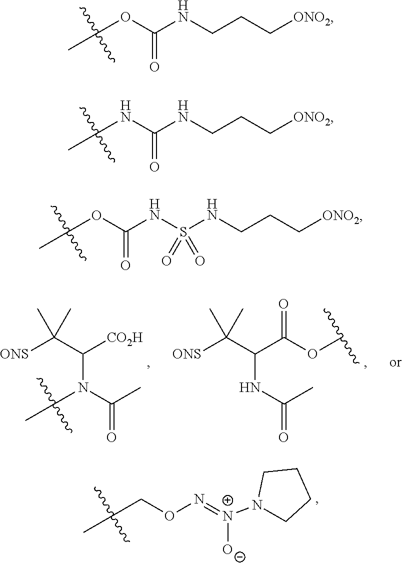
in which n is 1, 2, 3, 4, 5, 6 or 7;
Rₐ is H, C₁-C₁₀ alkyl, aryl, S(O)₂-aryl, CN, or CON(R_{b})₂; and each R_{b}, independently, is H or C₁-C₁₀ alkyl.

19. A kit comprising an NO-releasing moiety and an H₂S-releasing moiety covalently bonded together, a compound containing an NO-releasing moiety and an H₂S-releasing moiety covalently bonded to an aspirin derived core, or combinations thereof; and a solvent for dissolving the same, wherein compound or compounds are in dry form or dissolved in the solvent; and instructions for use of the compounds or compounds in dissolved form for treating seeds at their pre-germination stage.

20. The kit of claim 19, instructions for preparing an aqueous solution including the compounds or compounds in concentrations effective from treating the seeds.

21. The kit of claim 19, comprising a stock solution of the compound or compounds and instructions for diluting the stock solution with an aqueous medium to obtain an aqueous solution suitable for treating the seeds.
